(19) United States
(12) Patent Application Publication
(10) Pub. No.: US 2022/0072088 A1
(43) Pub. Date: Mar. 10, 2022

(54) REDUCING CLOSTRIDIUM PERFRINGENS VIRULENCE USING INHIBITORS OF AGR-LIKE QUORUM SENSING

(71) Applicants: The Regents of the University of California, Oakland, CA (US); University of Pittsburgh - Of the Commonwealth System of Higher Education, Pittsburgh, PA (US)

(72) Inventors: Francisco Uzal, San Bernardino, CA (US); Mauricio Andres Navarro Badilla, San Bernardino, CA (US); Bruce McClane, Pittsburgh, PA (US); Jihong Li, Pittsburgh, PA (US)

(21) Appl. No.: 17/470,775

(22) Filed: Sep. 9, 2021

Related U.S. Application Data
(60) Provisional application No. 63/075,966, filed on Sep. 9, 2020.

(51) Int. Cl. A61K 38/10; A61P 31/04; A23L 33/18; A23L 33/10; A23K 20/147; A23K 20/195; A23K 50/50

(52) U.S. Cl. CPC A61K 38/10; A61P 31/04; A23L 33/18; A23V 2002/00; A23K 20/147; A23K 20/195; A23K 50/50; A23L 33/127

(57) ABSTRACT

This disclosure provides materials and methods for reducing production of virulence factors in C. perfringens and or other pathogenic clostridia with related Agr-like QS systems using compounds that interfere with the Agr-like quorum sensing (QS) system. For example, Agr-like QS system inhibitors based on peptidomimetic compounds of the Agr-like QS system signaling peptide (SP) or the SP receptor of C. perfringens, and methods of using the Agr-like QS system inhibitors to prevent, treat, or ameliorate a disease associated with C. perfringens infection are provided.

### FEDERALLY SPONSORED RESEARCH OR DEVELOPMENT

This invention was made with government support under contract R21A1140010-02 awarded by the National Institute of Allergy and Infectious Disease. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 9, 2021, is named 7020_006 US1_SL.txt and is 10,301 bytes in size.

### BACKGROUND

Bacterial resistance to antimicrobial compounds has become a serious threat to human health and increases the spread of drug-resistant pathogens. As most antimicrobial compounds target essential bacterial physiological processes, these compounds exert selective pressure on bacteria and facilitate the emergence of resistant strains. Therapeutic strategies that circumvent the emergence and spread of multidrug-resistant pathogens are, therefore, urgently needed.

Pathogenic Clostridium species are generally resistant to multiple antibiotics, thanks to the slow growth of most species, obligate anaerobic life cycle, and their ability to form endospores, a non-metabolizing and durable cell form highly resistant to heat, desiccation, radiation, oxidation and numerous other chemical means of microbial control. Clostridium perfringens is an important cause of human infections, including traumatic gas gangrene and several common and serious illnesses originating in the intestines, most notably C. perfringens type F food poisoning, antibiotic-associated diarrhea, and enteritis necroticans. Recent studies identified a key role for the Accessory gene regulator (Agr)-like quorum sensing (QS) system in regulating C. perfringens toxin production. Since C. perfringens uses toxins to cause gas gangrene and intestinal infections, elucidating toxin production regulation in this bacterium is critical to understand its pathogenesis and develop novel therapeutic approaches.

Therapeutic strategies based on interfering with the Agr-like QS system may provide a means to ameliorate the symptoms of C. perfringens infections in humans and livestock. While the C. perfringens Agr-like QS system resembles the paradigm Staphylococcus aureus Agr QS system by involving a signaling peptide (SP), the C. perfringens SP substantially differs in sequence and length from the S. aureus SP. Another key difference is that C. perfringens does not encode a homolog of the receptor protein that binds SP in S. aureus. Thus, there is a need to identify specific inhibitors of one or more pathways of the Agr-like QS system in C. perfringens to prevent or reduce toxin production, reduce pathogen virulence and thereby provide alternatives to antibiotic interventions.

### SUMMARY

In general, the present disclosure provides materials and methods for inhibiting production of virulence factors in C. perfringens and/or other pathogenic clostridia with related Agr-like QS systems using compounds by interfering with the Agr-like quorum sensing (QS) system. For example, Agr-like QS system inhibitors such as peptidomimetic compounds of the signaling peptide (SP) and of a SP receptor of one or more strains of Clostridium perfringens are provided such as peptidomimetic compounds of the signaling peptide (SP) and of a SP receptor of one or more strains of Clostridium perfringens. The present disclosure is based in part on a new understanding of the pathogenesis of gas gangrene and identification of new therapeutic targets to prevent or ameliorate this disease and other diseases caused by C. perfringens. Because interference with virulence factors does not aim to eradicate the bacteria, the compounds and methods of the present disclosure do not exert a strong selective pressure on the bacteria and may decelerate the emergence and dissemination of resistant mutant strains.

Accordingly, in a first aspect, the present disclosure features a medicament comprising an Agr-like QS system inhibitor of Clostridium perfringens and a pharmaceutically acceptable carrier, wherein the inhibitor is a 6-R peptide, a C. perfringens Signaling Peptide (SP) receptor peptidomimetic, or a combination thereof. The Agr-like QS system inhibitor can include a VirS-based SP receptor peptidomimetic. The VirS-based SP receptor peptidomimetic can be a peptide having an amino acid sequence consisting of the sequence set forth in SEQ ID NO: 13 or SEQ ID NO: 10. The Agr-like QS system inhibitor can be in a salt, solvate, or prodrug form. The medicament can be formulated for oral, buccal, sublingual, nasal, intravenous, intramuscular, intrathecal, intraperitoneal, transdermal, or pulmonary administration. The medicament can include 25 to 100 μM of the Agr-like QS system inhibitor in a liquid carrier.

In a second aspect, the present disclosure features a food or animal feed comprising an Agr-like QS system inhibitor of Clostridium perfringens and an edible carrier, wherein the inhibitor is a 6-R peptide, a C. perfringens Signaling Peptide (SP) receptor peptidomimetic, or a combination thereof. The Agr-like QS system inhibitor can include a VirS-based SP receptor peptidomimetic. The VirS-based SP receptor can be a peptide having an amino acid sequence consisting of the sequence set forth in SEQ ID NO: 13 or SEQ ID NO: 10. The food or feed can include beef, poultry, gravies, or dried or pre-cooked food.

In a third aspect, the present disclosure features a method of preventing or treating a disease associated with a Clostridium perfringens infection or infection by other pathogenic clostridia having a Agr-like QS system comprising administering, to a subject in need of prevention or treatment, a therapeutically effective amount of an Agr-like QS system inhibitor of C. perfringens selected from a 6-R peptide, a C. perfringens Signaling Peptide (SP) receptor peptidomimetic, or a combination thereof (i.e., an Agr-like QS system inhibitor of C. perfringens selected from a 6-R peptide, a C. perfringens Signaling Peptide (SP) receptor peptidomimetic, or a combination thereof for the treatment of a disease associated with C. perfringens infection or infection by other pathogenic clostridia having a Agr-like QS system). The Agr-like QS system inhibitor can include a VirS-based SP receptor peptidomimetic. The VirS-based SP receptor peptidomimetic can be a peptide having an amino acid sequence consisting of the sequence set forth in SEQ ID NO: 13 or SEQ ID NO: 10. The subject can be selected from the group consisting of primates, rodents, domestic animals and game animals. The domestic animals can include sheep, cows, horses, pigs, or poultry. The inhibitor can be administered with a food selected from the group consisting of beef, poultry, gravies, and dried or pre-cooked foods. The inhibitor can be administered with an antibiotic effective for treating a Clostridium infection. The method can further include debridement or removal of necrotic tissue caused by the infection. The therapeutically effective amount is an amount effective for reducing production of at least one of alpha toxin (CPA), beta toxin (CPB), epsilon toxin (ETX), iota toxin (ITX), perfringolysin O (PFO), enterotoxin (CPE), NetB toxin (NetB) or beta2 toxin (CPB2) in an infected tissue of the subject as compared to production of the toxin in a corresponding tissue in an untreated subject infected with the bacteria. The therapeutically effective amount can be an amount that prevents or reduces swelling or hemorrhage in an infected tissue of the subject as compared to a corresponding tissue of an untreated subject infected with the bacteria. The therapeutically effective amount can be an amount that prevents or reduces at least one of muscle degeneration, necrosis and inflammation in an infected tissue of the subject as compared to a corresponding tissue of an untreated subject infected with the bacteria. The disease can be necrotic enteritis, gas gangrene, enterotoxemia, or enteritis. The inhibitor can be administered by intramuscular injection at a concentration of 25 to 100 μM.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale. The drawings illustrate generally, by way of example, but not by way of limitation, various embodiments discussed in the present document.

Reference is made to illustrative embodiments that are depicted in the figures, in which:

FIGS. 1A-C describe an agrB-null mutant of C. perfringens type A wild-type ATCC 3624. (A) PCR confirmation of the construction of an agrB-null mutant of wild-type ATCC 3624. (B) Southern blot hybridization of an intron-specific probe with DNA from wild-type ATCC 3624. The size of the DNA fragment, in kilobases (kb), is shown at the right. (C) RT-PCR evaluation of agrB expression. Reverse transcription of extracted RNA from each strain, followed by PCR, was performed. Expression of the agrB gene was demonstrated for the wild-type parent and the complementing strain, but no agrB transcription was detectable for the ATCC 3624 agrB-null mutant. All three ATCC 3624-related strains expressed similar levels of 16S RNA.

FIGS. 2A-B show a comparison of growth or alpha toxin (CPA) and perfringolysin O (PFO) production by wild-type ATCC 3624, the agrB mutant, and the complementing strain. (A) Cultures of each strain were grown to 24 h at 37° C. in TY medium. At the designated time points, the OD₆₀₀ of each culture was determined. Representative results of three repetitions for each strain are shown. (B) CPA and PFO Western blotting performed with the supernatant from the overnight culture of each strain grown overnight at 37° C. in TY medium. The molecular mass is indicated on the left. Representative Western blots for four repetitions are shown.

FIGS. 3A-D show virulence effects of agrB gene inactivation in a mouse model of C. perfringens type A-induced gas gangrene. (A) Macroscopic pathology showing changes in skeletal muscle of mice injected with wild-type ATCC 3624 and the complementing strain are characterized by swelling, edema, and hemorrhage. No gross lesions were observed in mice receiving the agrB mutant or DPBS buffer. (B) Microscopic pathology of the corresponding treatments show myriad rod-shaped bacteria (open arrow) in association with myonecrosis (solid arrow) in a mouse challenged with wild-type ATCC 3624 or the complementing strain but the absence of lesions in the skeletal muscle of mice receiving the agrB mutant or DPBS. (C) C. perfringens immunohistochemistry on the skeletal muscle of a mouse challenged with wild-type ATCC 3624 or the agrB mutant showing large numbers of brown, short rods (black arrows). Panels A to C show representative results for eight mice receiving each treatment. (D) Histological score of skeletal muscles treated with the indicated inocula for 4 h. Error bars show the standard errors of the means. *, P<0.05. Scale bar, 25 μm.

FIG. 4 shows recovery of viable C. perfringens from challenged tissues. After 4 h of incubation, samples of skeletal muscle from mice challenged with ˜10⁶ CFU of wild-type ATCC 3624, the agrB mutant, the complementing strain, and DPBS only. Error bars show standard errors of the means. ns, not significant. Each bar represents the mean results using samples from eight mice.

FIGS. 5A-C shows the effects of the 6-R synthetic signaling peptide on in vitro CPA or PFO production by ATCC 3624. (A) Representative CPA and PFO Western blots performed with supernatants from overnight cultures of the wild-type ATCC 3624, coincubated with three different concentrations of the 6-R peptide in TY medium grown overnight at 37° C. The molecular mass is indicated on the left. (B) CPA and PFO production levels in the presence of the 6-R peptide (100 μM) compared to the wild-type levels using ImageJ densitometric analyses of three separate Western blots. (C) Cultures of the ATCC 3624 strain with or without 6-R peptide (100 μM) were grown to 24 h at 37° C. in TY medium. At the designated time points, the OD₆₀₀ of each culture was determined. Representative results of three repetitions for each strain are shown. Error bars show the standard errors of the means. *, P<0.05. The results shown in panel A are representative of three Western blot experiments for each toxin, and panels B and C show the mean of three repetitions.

FIGS. 6A-D show inhibitory effects of the 6-R peptide on the virulence of ATCC 3624 in a mouse model of C. perfringens type A-induced gas gangrene. (A) Macroscopic pathology of mice injected with the corresponding treatment. (B) Microscopic pathology of mice injected with the corresponding treatment showing the severe myonecrosis in a mouse receiving the ATCC 3624 strain only. Only a few inflammatory cells (neutrophils) are seen between the muscle fibers in a mouse receiving the 6-R peptide (100 μM). (C) Histological scores of skeletal muscles treated with the indicated inocula for 4 h. (D) Comparison of growth yields by C. perfringens type A ATCC 3624 strain co-incubated with or without the 6-R peptide (100 μM) recovered from challenged skeletal muscle of mice. Error bars show standard errors of the means. *, P<0.05; ns, not significant. The data in the panels are representative of eight mice each. Scale bar, 50 μm.

FIGS. 7A-B illustrate a model for inhibition of binding of C. perfringens Natural Signaling Peptide (SP) 5R (SEQ ID NO: 1) by the decoy peptide KIGK (SEQ ID NO: 10). (A) depicts the binding of the SP to the second extracellular loop of VirS (amino acids 111 to 124 of C. perfringens strain CN1795) and subsequent phosphorylation and activation of VirR (FIG. 7A discloses “RVDIGI” as SEQ ID NO: 14); (B) shows the proposed mechanism of virulence attenuation in the presence of the decoy peptide. FIG. 7B discloses SEQ ID NOS 1 and 14, respectively, in order of appearance. Without being bound by theory, there is evidence to suggest that the structures of certain autoinducing peptides (AIPs) believed to exist as thiolactones, such as the 5-mer thiolactone ring initially produced by C. perfringens, may be cysteine-containing homodetic peptides in their functional state.

FIGS. 8A-C show growth curves and timing of CPB production vs. virS, agrD and cpb expression in wild-type CN1795 and CN3685. (A) Growth curves for wild-type CN1795 and CN3685 cultured at 37° C. for up to 24 h in TY medium. Aliquots of each culture were measured for their OD₆₀₀ at 1 h, 3 h, 5 h, 8 h, 10 h and 24 h. This experiment was repeated three times and a representative result is shown. (B) Quantitative RT-PCR (qRT-PCR) analyses of cpb expression levels (left) and CPB Western blot analysis (right) for CN1795 and CN3685. Transcript levels were determined using 10 ng of RNA isolated from TY cultures of these strains grown for 1 h, 3 h, 5 h, 8 h, 10 h and 24 h at 37° C. Average C_{T} values were normalized to that of the 16S rRNA housekeeping gene and fold differences were calculated using the comparative C_{T} method (2^{-ΔΔCT}). Values of each bar indicate the fold change versus 1 h culture. Western blot analysis for CPB production in supernatants of the two wild-type strains cultured in TY culture for 1 h, 3 h, 5 h, 8 h, 10 h and 24 h; (C) qRT-PCR analyses of agrD and virS expression levels using the same cDNA preparation as used for measuring cpb transcripts. qRT-PCR analyses shown in panels B and C were repeated three times, and mean values are shown. The error bars indicate standard deviations. A representative CPB Western blot based upon 3 repetitions is shown.

FIGS. 9A-B show modeling of the VirS protein produced by CN1795 versus CN3685. (A) Comparison of signaling sensitivity of CN1795 and CN3658 to SP-based peptides 5R (a thiolactone ring with SEQ ID NO: 1) and 8R (a thiolactone ring with a tail SEQ ID NO: 42). CPB production (left panel) by wild type or agrB null mutant (agrBKO) strains of CN3685 and CN1795 in the presence of 100 μM concentration of 5R and 8R (structures shown in the right panel). The samples for CPB Western blotting were prepared from the supernatants of overnight TY cultures (about 16 h) at 37° C. (B) The predicted topology of VirS protein produced by two wild-type strains. The predicted CN1795 VirS structure is shown in black numbers (total 440 amino acids). The predicted CN3685 VirS structure is shown in red numbers (total 446 amino acids). * with different colors in original to show the difference in this VirS region between these two strains (including the fragment set forth as SEQ ID NO: 14 (RVDIGI)) Amino acid (Aa) differences indicated by * between the two strains are shown on the right side, with amino acid sequence numbers for CN1795 shown in black and those of CN3685 shown in red in original.

FIGS. 10A-D describe the preparation and characterization of CN1795 or CN3685 agrB/virS double null mutants and complementation of those double mutants to express a swapped VirS. (A) PCR confirmation of agrB/virS double null mutant construction by targeted intron-based mutagenesis and genetic complementation of those mutants with a swapped virR/S operon. Using DNA isolated from wild-type strains, a PCR assay amplified ˜500 bp products using internal agrB primers (upper panel) or internal virS primers (lower panel). After targeted insertion of a 900 bp intron, the same PCR assays amplified an ˜1.5 kb product using DNA isolated from the double null mutant (DKO) strains. After complementation of the double mutants to carry the swapped virR/S operon (creating CN1795DKOc3685virR/S and CN3685DKOc1795virR/S), agrB PCR products remained the large size indicative of an intron insertion, but the virS PCR products were the smaller size present in wild-type strains, confirming genetic virS complementation. A 1 kb molecular ladder (Fisher Scientific) was used, with the size in bp shown at left. (B) Southern blot hybridization of an intron-specific probe with DNA from wild-type strains, the agrB single mutants or agrB/virS double null mutants. DNA from each strain was digested with EcoRI and electrophoresed on a 1% agarose gel prior to blotting and hybridization with an intron-specific probe. Size of DNA fragments, in kilobases (kb) is shown at left. (C) RT-PCR analyses for expression of 16S RNA (top panel), the agrB gene (middle panel) or the virS gene (bottom panel) by wild-type CN1795 (left panel) or CN3685 (right panel), their agrB/virS double null mutants (DKO), or complementing strains of those double mutants with a swapped virR/S operon. Indicating that the RNA preparations from all strains were free from DNA contamination, those samples did not amplify a product when subjected to PCR without reverse transcription (data not shown) (D) Western blot analyses of CPB production by wild-type strains, agrB/virS double null mutants or those mutants cultured in the presence of 100 μM 5R or 8R peptides. Size of proteins in kDa is shown at left. All experiments were repeated three times and representative results of three repetitions are shown.

FIGS. 11A-B show western blot analyses of CPB production by (A) CN1795 and (B) CN3685 wild-type strains, agrB/virS double mutant strains, and swapped complementing strains grown in the presence or absence of 100 μM of the 5R or 8R peptide. Cultures were grown for 5 h at 37° C. in TY medium and the OD₆₀₀ values of each culture were adjusted to the same density. Supernatants were then collected and equal volumes were subjected to CPB Western blotting. Size of proteins in kDa is shown at right. All experiments were repeated three times and results representative of three repetitions are shown.

FIGS. 12A-D describe the preparation and characterization of CN1795 or CN3685 virS null mutants and complemented strains. (A) PCR confirmation of virS null mutants created by intron-based mutagenesis or complementing strains carrying a virR/S operon from CN1795 or CN3685 (indicated by cCN1795virR/S or cCN3685virR/S). Using DNA isolated from wild-type strains, an ˜500 bp product was PCR amplified using internal virS primers. However, after targeted insertion of a 900 bp intron into virS, the same PCR assay amplified an ˜1.5 kb product using DNA from the null mutant strains. Using DNA from the complemented strains, the same-size virS PCR products were amplified as when using DNA from wild-type strains. A 1 kb molecular ladder (Fisher Scientific) was also electrophoresed and the size in bp is shown at left. (B) Southern blot hybridization of an intron-specific probe with DNA from wild type or the virS null mutants of CN1795 or CN3685. DNA from each strain was digested with EcoRI and then electrophoresed on a 1% agarose gel prior to blotting and hybridization with an intron-specific probe. Size of DNA fragments, in kilobases (kb), is shown at right. (C) RT-PCR analyses of 16S RNA (top panel), and virS (bottom panel) transcription in wild-type CN1795 (left panel) or CN3685 (right panel), their virS null mutants, or complementing strains carrying the virR/S operon of either strain. To show that the RNA preparations from both strains were free from DNA contamination, the samples were also subjected to PCR without reverse transcription, but no products were amplified (data not shown). (D) Western blot analyses of CPB expression by wild type, virS null mutants and complementing strains. Size of proteins in kDa is shown at right. All experiments were repeated three times and results representative of three repetitions are shown.

FIGS. 13A-B show Strepavidin beads containing bound B-5R pull down His₆-tagged (SEQ ID NO: 43) VirS. (A) CPB production by wild-type or agrB mutant CN1795 (upper panel), wild type or agrB mutant CN3685 (lower panel) or those null mutants incubated in the presence of a 100 μM concentration of B-5R. Size of proteins in kDa is shown at left. (B) His₆-tag (SEQ ID NO: 43) Western blot of B-per buffer extracts of CN3685::virSc3685virR/Shis (lane 1) or CN3685::virSc3685virR/S (lane 3). After those extracts were incubated with streptavidin beads preincubated with B-5R or 5R, unbound supernatant material is shown in lanes 2 and 4. Pull-downs of extracts from complementing strains using beads preincubated with 5R or B-5R are shown in lanes 5 to 9. Note in these lanes that using CN3685::virSc3685virR/Shis-containing extracts, but not CN3685::virSc3685virR/S-containing extracts, incubation of beads pretreated with B-5R, but not 5R, resulted in pull-down of a protein that reacted with His₆-tag (SEQ ID NO: 43) antibody and was ˜50 kDa, the size of VirS. Size of protein markers in kDa is shown at left. All experiments were repeated three times and results shown are representative of three repetitions.

FIGS. 14A-C show the KIGK peptide (SEQ ID NO: 10) corresponding to the major predicted VirS ECL2 inhibits the ability of the 5R SP to induce CPB production. (A) Western blot showing effects of KIGK preincubation with 5R on CPB production by agrB null mutant strains of CN1795 and CN3685. Cultures of these strains were grown for 5 h at 37° C. in TY medium with DMSO, DMSO plus 5R (100 μM) or DMSO plus 5R (100 μM) that had been preincubated with KIGK (500 μM). (B) Western blot showing effects of preincubation of 5R with the KIGK_D peptide (SEQ ID NO: 12), which corresponds to the mutated VirS second ECL, on CPB production by agrB null mutant strains of CN1795 and CN3685. Cultures of those strains were grown for 5 h at 37° C. in TY medium with DMSO, DMSO plus 5R (100 μM) or DMSO plus 5R (100 μM) that had been preincubated with KIGK_D (500 μM). (C) Western blot showing effects of KIGK on CPB production by wild-type CN1795 and CN3685 cultures. Cultures of those strains were grown in TY medium with DMSO, DMSO plus KIGK (1 mM) or DMSO plus KIGK_D (1 mM) for 5 h at 37° C. For all panels, CPB Western blots of culture supernatants are shown. Size of proteins in kDa is shown at left. All experiments were repeated three times and the results representative of three repetitions are shown.

FIGS. 15A-B show qRT-PCR analysis for virS and agrD gene expression in CN1795 (left) or CN3685 (right) wild-type, agrB or virS null mutants, and complemented strains. qRT-PCR analyses of agrD (A) and virS (B) transcript levels were performed with 20 ng of the RNA isolated from 5-h (for agrD analyses) or 2-h (for virS analyses) TY medium cultures of the wild-type, virS or agrB null mutants and their complemented strains (comp.). Average C^{T} values were normalized to the value for the housekeeping 16S RNA gene, and the fold differences were calculated using the comparative C_{T};2^{-ΔΔCT} method. The value of each bar indicates the calculated fold change relative to the value for the wild-type strains. Shown are the mean values from three independent experiments. *, P<0.05 compared to the wild-type culture by ordinary one-way ANOVA.

FIG. 16 shows the KIGK peptide (but not the KIGK_D negative control peptide) can reduce the ability of ATCC3624 to cause gas gangrene in the mouse model. Tissue treated with the indicated inocula for 4 h prior to challenge. Each group included 10 mice. Error bars show the standard errors of the means. *, P<0.05.

### DETAILED DESCRIPTION

The present disclosure describes materials and methods for inhibiting production of virulence factors in C. perfringens and or other pathogenic clostridia with related Agr-like QS systems. For example, Agr-like QS system inhibitors such as peptidomimetic compounds of the signaling peptide (SP) or the SP receptor of one or more types or strains of C. perfringens, compositions for administration and consumption that include these Agr-like QS system inhibitors, and methods of treatment or prevention of diseases caused by infection of C. perfringens and or other pathogenic clostridia, such as gas gangrene, necrotic enteritis, and food poisoning, using these compositions are provided.

### Definitions

The terms recited below have been defined as described below. All other terms and phrases in this disclosure shall be construed according to their ordinary meaning as understood by one of skill in the art.

The term “Agr-like QS system inhibitor” refers to a compound that inhibits or attenuates Agr-like QS system regulated production of a toxin by the bacterium belonging to the genus Clostridium. The Agr-like QS system inhibitors of the present disclosure are identified by the structure activity relationship (SAR) of the signaling peptide (SP) and non-native mimetics of the peptide or its receptor that are capable of inhibiting toxin production in C. perfringens and or other pathogenic clostridia with related Agr-like QS systems.

The “6-R Peptide” is a synthetic peptide characterized as a six-membered thiolactone ring containing the natural five amino acids of the C. perfringens signaling peptide (SP) plus an adjacent amino acid histidine (CLWFTH (SEQ ID NO: 2)) as shown below:

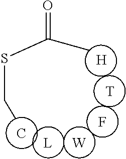

The “KIGK peptide” is a synthetic peptide having the amino acid sequence as set forth in SEQ ID NO: 10 (i.e., KINSLVNVSELLGK).

The term “subject” refers to a human or animal, including a primate, rodent, domestic animal and game animal. Domestic and game animals include livestock, for example, cows, horses, pigs, deer, bison, buffalo, poultry (e.g., turkey and chicken), emu, ostrich, canine and feline species. In some embodiments, the subject is a mammal or avian. The terms, “individual,” “patient” and “subject” are used interchangeably in the present disclosure.

As used herein, the terms “treat,” “treatment,” “treating,” or “amelioration” refer to therapeutic treatments, the object of which is to reverse, alleviate, ameliorate, inhibit, slow down or stop the progression or severity of a condition associated with clostridial infection. The term “treating” includes reducing or alleviating at least one adverse effect or symptom of a clostridial infection. The term “treatment” of a clostridial infection also includes providing relief from the symptoms or side-effects of the infection (including palliative treatment).

Treatment is generally “effective” if one or more symptoms or clinical markers are reduced. Alternatively, treatment is “effective” if the progression of a clostridial infection is reduced or halted. Thus, treatment includes not just the improvement of symptoms or markers, but also include a cessation or at least slowing of progress or worsening of symptoms that would be expected in absence of treatment. Beneficial or desired clinical results include, but are not limited to, alleviation of one or more symptom(s) of an infection, diminishment of extent of an infection, stabilized (i.e., not worsening) state of an infection, delay or slowing of progression of an infection, amelioration or palliation of the infection, and remission (whether partial or total), whether detectable or undetectable. The amount delivered can be therapeutically effective despite a lack of significant change in the number of viable bacteria. Thus, an “effective amount” indicates the amount of an active ingredient that is at least necessary to achieve a desired effect, e.g., inhibition of virulence factors, and prevention and/or treatment of pathological changes caused by a Clostridium bacterium. For example, the amount delivered (e.g., administered or consumed) is effective if amount of toxin detected in the subject or tissue thereof, is reduced (or inhibited) compared to the amount of toxin produced in the absence of treatment or in response to a sham treatment. The toxin reduced or inhibited can be the alpha toxin (CPA), beta toxin (CPB), epsilon toxin (ETX), iota toxin (ITX), perfringolysin O (PFO), enterotoxin (CPE), NetB toxin (NetB), or beta2 toxin (CPB2). The amount delivered (e.g., administered or consumed) is also effective if the gross pathological changes associated with infection are reduced (e.g., comparatively reduced swelling or discoloration in infected muscle tissue), and/or microscopic pathological changes associated with infection are reduced (e.g., no or less severe muscle degeneration, necrosis and/or inflammation, no or relatively fewer gram positive intralesional rods observed, no or minimal change in blast cell count, no or minimal change in tissue perfusion).

The terms “virulence”, “virulent” and variations thereof refer to a pathogen's ability to cause symptoms or signs of macroscopic or microscopic pathological changes in a subject.

“Gas gangrene” or “clostridial myonecrosis” refers to a necrotizing infection of subcutaneous tissue and muscle previously referred to as malignant edema caused by Clostridium chauvoei, C. septicum, C. novyi type A, C. perfringens type A, and C. sordellii, acting singly or in combination. A presumptive diagnosis of gas gangrene can be established based on clinical history, clinical signs, and gross and microscopic changes, identification of the clostridia involved is required for confirmatory diagnosis. The disease is characterized by fever, pain, massive local edema, gas production, and severe muscle tissue destruction, and it often develops into systemic toxemia, shock, sepsis, or death, which occurs in more than 50% of cases.

The terms “decrease”, “reduction”, “inhibition”, or variations thereof refer to a decrease by a statistically significant amount, such as by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more (e.g., a complete inhibition or reduction as compared to an appropriate control).

Clostridium infections, particularly C. perfringens infections, can affect various parts of the body to cause gastroenteritis, muscle necrosis and soft-tissue infection, which is characterized by crepitant cellulitis, myositis, and clostridial myonecrosis, skin and tissue necrosis, and mild wound infections. Hospital-acquired clostridial infection can occur in postoperative and immunocompromised patients to cause severe clostridial sepsis. Agr-like QS system inhibitors of the present disclosure are useful in the treatment of such infections and can reduce the virulence thereof. Agr-like QS system inhibitors can be employed in human treatment or in veterinary treatment.

Embodiments of the present disclosure describe a method of treating or preventing various significant systemic and enteric diseases, in both humans and animals associated with bacterium of the genus Clostridium, including gas gangrene (Clostridial myonecrosis), food poisoning and non-foodborne diarrhea, and enterocolitis including administering or feeding an effective amount of an Agr-like QS system inhibitor, or salt thereof, to a subject in need of treatment or at risk of infection.

Embodiments of the present disclosure also include contacting a C. perfringens bacterium or other pathogenic clostridia with related Agr-like QS systems with a virulence reducing amount of a Agr-like QS system inhibitor, or salt thereof. The contact can occur in vitro (e.g., culturing the bacteria in the presence of the inhibitor).

An Agr-like QS system inhibitor of the present disclosure can be a peptidomimetic compound of the signaling peptide (SP) of the Clostridium bacterium. The SP peptidomimetic compound can be capable of inhibiting SP receptor activation, and thereby inhibiting toxin production. A SP peptidomimetic can be the 6-R Peptide. There are several strategies known to the skilled artisan for synthesizing cyclic peptide compounds including scaffold-based and enzyme-based approaches.

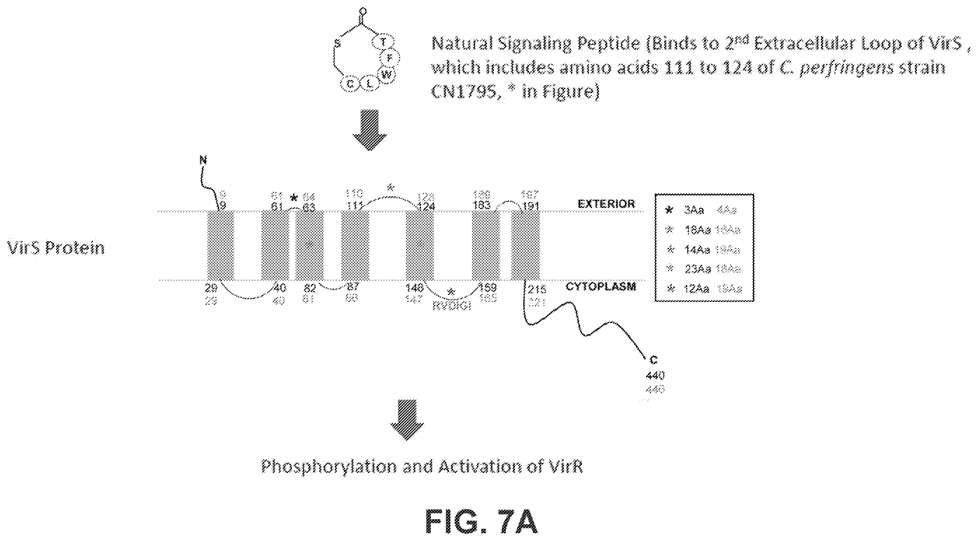

An Agr-like QS system inhibitor can be a SP receptor decoy or a peptidomimetic compound of a SP receptor capable of interfering with SP binding to any binding partner, SP binding to VirS, VirS phosphorylation of VirR, and/or VirR activation. The SP receptor decoy can be a peptidomimetic of an extracellular loop of a VirS protein expressed by a Clostridium bacterium. FIG. 7A-B show three predicted extracellular loops (ECLs). Thus, in some embodiments, the Agr-like QS system inhibitor of the present disclosure is based on the VirS first ECL, the VirS second ECL or the VirS third ECL, for any functional variant of a VirS protein expressed by a Clostridium bacterium. The VirS protein can be expressed by any type of Clostridium strain (e.g., one or more of toxinotypes A-G). For example, the VirS protein can be expressed in a type G strain causing avian necrotic enteritis, CPE-producing type F strains causing human food poisoning and CPA/PFO-producing type A strains causing gas gangrene. In some cases, Agr-like QS system inhibitor has the sequence set forth as SEQ ID NO: 13 or SEQ ID NO: 10.

Agr-like QS system inhibitors of the present disclosure can be used to attenuate Clostridium virulence mechanisms alone or in combination. For example, one or more SP peptidomimetic compounds can be used with one or more SP receptor peptidomimetic compounds. The inhibitors can be co-formulated in a unit dosage form or a food or feed product, or formulated separately and administered together (sequentially or simultaneously).

The Agr-like QS system inhibitors of the present disclosure may contain chemical groups (acidic or basic groups) that can be in the form of salts. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Salts of the Agr-like QS system inhibitor include “pharmaceutically acceptable salts” which refers to those salts which retain the biological effectiveness and properties of the free bases or free acids, and which are not biologically or otherwise undesirable. Pharmaceutically acceptable salts comprise pharmaceutically-acceptable anions and/or cations.

The Agr-like QS system inhibitors, and salts thereof, may exist in their tautomeric form, in which hydrogen atoms are transposed to other parts of the molecules and the chemical bonds between the atoms of the molecules are consequently rearranged. All tautomeric forms, insofar as they may exist, are included within the invention. Additionally, where the Agr-like QS system inhibitors have trans and cis isomers and may contain one or more chiral centers, the present disclosure includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The Agr-like QS system inhibitors may be in the free or hydrate form.

With respect to the various Agr-like QS system inhibitors described, the atoms therein may have various isotopic forms, e.g., isotopes of hydrogen include deuterium and tritium. All isotopic variants of compounds of the invention are included within the invention and particularly included at deuterium and ¹³C isotopic variants. Such isotopic variants may be useful for carrying out various chemical and biological analyses, investigations of reaction mechanisms and the like. Methods for making isotopic variants are known in the art.

The disclosure expressly includes pharmaceutically usable solvates of Agr-like QS system inhibitors. Solvation can occur in the course of the manufacturing process or can take place, e.g. as a consequence of hygroscopic properties of an initially anhydrous compound, or the compound can be prepared as a solvate to improve its stability for large-scale production.

Agr-like QS system inhibitors of the present disclosure can have prodrug forms. Any compound that will be converted in vivo to provide a biologically, pharmaceutically, or therapeutically active form of a Agr-like QS system inhibitor is a prodrug. Prodrugs of the Agr-like QS system inhibitors can be useful in the methods of treatment. Various examples and forms of prodrugs are well known in the art.

The effective amount of an Agr-like QS system inhibitor can be administered in a pharmaceutical composition (e.g., a dosage form). The pharmaceutical composition may be administered according to any administration route considered to be appropriate by a skilled person in the art. For example, the pharmaceutical composition may be administered orally, buccally, sublingually, nasally, intravenously, intramuscularly, intrathecally, intraperitoneally, transdermally (for example, as an ointment or a patch), or by pulmonary administration.

Any suitable form of administration can be employed in the method herein. The Agr-like QS system inhibitor can, for example, be administered in oral dosage forms including tablets, capsules, pills, powders, granules, elixirs, tinctures, suspensions, syrups and emulsions. Dosage forms may include sustained release or timed release formulations. Agr-like QS system inhibitor can also be administered in intranasal form by topical use of suitable intranasal vehicles. For intranasal or intrabronchial inhalation or insulation, the Agr-like QS system inhibitor may be formulated into an aqueous or partially aqueous solution, which can then be utilized in the form of an aerosol.

The dosage requirements need to achieve the “therapeutically effective amount” vary with the particular formulations employed, the route of administration, and clinical objectives. Based on the results obtained in standard pharmacological test procedures, projected daily dosages of active compound can be determined as is understood in the art. The therapeutically effective amount of a given compound or formulation will depend at least in part upon, the mode of administration (e.g., intravenous, oral, topical administration), any carrier or vehicle employed, and the subject to whom the formulation is to be administered (age, weight, condition, sex, etc.). In some cases, the Agr-like QS system inhibitor is administered parenterally (e.g., by intramuscular injection) at a concentration of about 25 to 100 μM, about 40 to 100 μM, about 75 to 100 μM or about 100 μM. The volume administered depends on the subject.

The pharmaceutical composition of the present invention contains a pharmaceutically acceptable carrier depending on each of those administration routes. Pharmaceutically acceptable carriers are those carriers that are compatible with the other ingredients in the formulation and are biologically acceptable. Carriers can be solid or liquid. Solid carriers can include one or more substances that can also act as flavoring agents, lubricants, solubilizers, suspending agents, fillers, glidants, compression aids, binders, tablet-disintegrating agents, or encapsulating materials. Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators.

Compositions for oral administration can be in either liquid or solid form. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidone, low melting waxes and ion exchange resins. The carrier can also be in the form of creams and ointments, pastes, and gels. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable.

Suitable examples of liquid carriers for oral and parenteral administration include water of appropriate purity, aqueous solutions (particularly containing additives, e.g. cellulose derivatives, sodium carboxymethyl cellulose solution), alcohols (including monohydric alcohols and polyhydric alcohols e.g. glycols) and their derivatives, organic solvents (DMSO) and oils. For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant. Liquid pharmaceutical compositions that are sterile solutions or suspensions can be administered by, for example, intramuscular, intraperitoneal or subcutaneous injection. Sterile solutions can also be administered intravenously.

Agr-like QS system inhibitors can be co-administered or co-formulated with antibiotics used for the treatment of clostridial infections. One of ordinary skill in the art can select form a variety of known antibiotics, which may be used alone or in combination, and which can include broad-spectrum intravenous antibiotics. For example, one or more Agr-like QS system inhibitors can be used in combination with intravenous or oral antibiotics.

Administration of one or more the Agr-like QS system inhibitors can be combined with another form of treatment, such as a surgical interventions or hyperbaric oxygen treatment. For example, one or more Agr-like QS system inhibitors can be used in combination with debridement or removal of the necrotic tissue (e.g., amputation).

In another embodiment, the disclosure provides a medicament for treatment of a clostridial infectious disease, particularly an infection by C. perfringens. The medicament includes a therapeutically effective amount of one or more Agr-like QS system inhibitors. The medicament can also include a therapeutically effective amount of one or more antibiotics. The medicament may be an oral dosage form, an intravenous dosage form, an intramuscular dosage form or any other art-recognized dosage form.

The present disclosure also includes a method for making the medicament which includes combining a therapeutically effective amount of one or more Agr-like QS system inhibitors with a pharmaceutically acceptable carrier. The carrier can be selected from known excipients as appropriate for a given method of administration. The method for making the medicament can further include combining a therapeutically effective amount of one or more antibiotics in the medicament.

The effective amount of an Agr-like QS system inhibitors can be administered in a food or animal feed product. The food or animal feed product containing an Agr-like QS system inhibitor of the present disclosure can inhibit the toxin production by the bacteria in the subject after consumption and also in a feed or food product before it is consumed (i.e., during storage), which is advantageous as Clostridium bacteria form a heat-resistant spore that is not easily sterilized by heating during food or feed packaging. Therefore, embodiments of the present disclosure also include methods of preventing or reducing the effects of food poisoning associated with Clostridium spoilage of food or feed. Beef, poultry, gravies, and dried or pre-cooked foods are common sources of C. perfringens infections. C. perfringens infection often occurs when foods are prepared in large quantities and kept warm for a long time before serving. Outbreaks often happen in institutions, such as hospitals, school cafeterias, prisons, and nursing homes, or at events with catered food. The inhibitor can be added to the food directly, or added in combination with an edible carrier. An effective amount can be an amount that is sufficient to prevent the development of diarrhea and/or abdominal cramps in an adult after eating contaminated food or minimizing the duration or severity of symptoms after eating contaminated food. Prevention includes, for example, the absence of clinical symptoms of C. perfringens infection within 24 hours of eating C. perfringens contaminated food.

The Agr-like QS system inhibitor can be combined with an edible carrier. The food or animal feed product can be a liquid product (solution, suspension, or emulsion), semi-moist product, powder, or a solid-molded or extruded product. The effective amount of the inhibitor can be 0.001 to 50% by weight or about 0.01 to 10% by weight relative to dry weight of the food or animal feed product.

The animal feed can be given to a non-human animal including a pet or companion animals, laboratory animals (e.g., mouse and rat); poultry such as chicken, quail, turkey, duck, goose, pheasant, Guinea fowl, and squab; livestock such as swine, cow, horse, sheep, and goat; and fish and shellfish, such as salmon, sweet fish, tuna, yellowtail, flounder, seabream, eel, and shrimp raised in aquaculture.

Embodiments of the present disclosure include methods preventing, inhibiting, and/or treating C. perfringens infection or infection by other pathogenic clostridia with related Agr-like QS systems in non-human animals by feeding the animal feed to the animal. For example, one or more embodiments of the present disclosure includes preventing, inhibiting, and/or treating Clostridium perfringens-associated necrotic enteritis in poultry (e.g., chicken) by feeding to poultry in need thereof an animal feed that includes an effective amount of an Agr-like QS system inhibitor.

The following Examples are intended to illustrate the above invention and should not be construed as to narrow its scope. Variations and modifications may be made while remaining within the scope of the invention.

### EXAMPLES

### Example 1. The Agr-Like Quorum-Sensing System is Important for Clostridium Perfringens Type A Strain ATCC 3624 to Cause Gas Gangrene in a Mouse Model

Clostridium perfringens type A is involved in gas gangrene in humans and animals. Following a traumatic injury, rapid bacterial proliferation and exotoxin production result in severe myonecrosis. C. perfringens alpha toxin (CPA) and perfringolysin O (PFO) are the main virulence factors responsible for the disease. Recent in vitro studies have identified an Agr-like quorum-sensing (QS) system in C. perfringens that regulates the production of both toxins. The system is composed of an AgrB membrane transporter and an AgrD peptide that interacts with a two-component regulatory system in response to fluctuations in the cell population density. In addition, a synthetic peptide named 6-R has been shown to interfere with this signaling mechanism, affecting the function of the Agr-like QS system in vitro. In the present example, C. perfringens type A strain ATCC 3624 and an isogenic agrB-null mutant were tested in a mouse model of gas gangrene. When mice were intramuscularly challenged with 10⁶ CFU of wild-type ATCC 3624, severe myonecrosis and leukocyte aggregation occurred by 4 h. Similar numbers of an agrB-null mutant strain produced significantly less severe changes in the skeletal muscle of challenged mice. Complementation of the mutant to regain agrB expression restored virulence to wild-type levels. The burdens of all three C. perfringens strains in infected muscle were similar. In addition, animals injected intramuscularly with wild-type ATCC 3624 coincubated with the 6-R peptide developed less severe microscopic changes. This study provides the first in vivo evidence that the Agr-like QS system is important for C. perfringens type A-mediated gas gangrene.

### INTRODUCTION

Clostridium perfringens is a Gram-positive, anaerobic, spore-forming bacterium that is responsible for a number of human and animal diseases due to the production of several toxins. Toxin production patterns vary among individual strains. This variability permits a classification system that assigns C. perfringens isolates to one of seven types (A to G) based upon the presence of the alpha (CPA), beta (CPB), epsilon (ETX), iota (ITX), enterotoxin (CPE), and necrotic enteritis B-like (NetB) toxin genes.

C. perfringens type A is the main cause of clostridial myonecrosis (gas gangrene) in humans and animals. The disease commonly starts with the infection of soft tissue, particularly muscle, by C. perfringens spores or vegetative cells as a result of a traumatic injury. Gas gangrene is clinically characterized by pain, fever, local edema, gas production, and necrosis of skeletal muscle, usually progressing to toxemia, shock, sepsis, and often death. The main virulence factor of C. perfringens for producing gas gangrene is CPA, a toxin with phospholipase C and sphingomyelinase activities that is encoded by the cpa (plc) gene. In addition, the pore-forming toxin perfringolysin O (PFO), encoded by the pfoA gene, acts synergistically with CPA during the pathogenesis of gas gangrene. CPA and PFO alter the extravasation of inflammatory cells, decreasing the infiltration of such cells to the site of infection. Both toxins have also been shown to induce upregulation of adhesion molecules on the surface of inflammatory cells, which would promote intravascular cell aggregation, followed by vascular occlusion.

Bacterial pathogens often regulate their virulence gene expression in response to environmental signals. This regulation commonly involves two, sometimes cross-talking, regulatory systems named two-component regulatory systems (TCRS) and quorum-sensing (QS) systems. QS systems control gene expression in response to bacterial population density through the production and detection of autoinducing peptides (AIPs), a group of small extracellular signaling molecules that sometimes bind to and activate the membrane sensor component of a TCRS.

The accessory gene regulator (Agr) system in Staphylococcus aureus is a prototype regulatory system involving both TCRS and QS systems. It consists of four cotranscribed genes: agrB, agrD, agrC, and agrA. The agrD gene encodes the AIP, which is processed to the active form by the AgrB transporter and then secreted extracellularly. Once a sufficient concentration of the AIP accumulates in the extracellular environment to trigger activation of the AIP-binding AgrC membrane sensor, the AgrC/AgrA TCRS then regulates gene expression.

Similar Agr-like regulatory systems are present in other Gram-positive pathogens, including C. perfringens. The C. perfringens genome carries an Agr-like operon encoding both an AgrD peptide and an AgrB membrane transporter. This Agr-like operon is highly conserved among C. perfringens strains but, at least for predicted Signaling Peptides (SPs), is quite different from the Agr-like QS systems present in other pathogenic clostridia. However, unlike S. aureus, the Agr-like QS locus of C. perfringens does not appear to directly encode a TCS. Instead, it has been proposed that the VirR/VirS TCRS often performs this function based upon observations that expression of several C. perfringens toxin genes, including those encoding CPA, PFO, CPB and NetB, are controlled by both the Agr-like QS system and the VirR/VirS TCRS.

Another difference between the Agr systems of S. aureus and C. perfringens concerns their respective AIPs and SPs. The S. aureus AIP, which varies among strains, is a 7- to 9-amino-acid peptide containing a five-member thiolactone ring with a short amino acid tail. Without being bound by theory, there is evidence to suggest that the structures of certain AIPs believed to exist as thiolactones, such as the 5-mer thiolactone ring initially produced by C. perfringens, may be cysteine-containing homodetic peptides in their functional state. In contrast, the SP in the C. perfringens Agr-like QS system is likely a tailless five-member thiolactone ring whose amino acid sequence differs from the thiolactone ring in the S. aureus AIP. Interfering with AIP signaling can affect Agr-like QS regulation. A synthetic peptide named 6-R, which is a six-membered thiolactone ring containing the natural five amino acids of the C. perfringens SP plus an adjacent amino acid, reduces production of CPB by some C. perfringens type B and type C strains.

Since agrB- or agrD-null mutants of C. perfringens are impaired for the in vitro production of CPA and PFO and both of these toxins are important for type A strains to cause gas gangrene, the present study directly investigated whether the Agr-like QS system regulates the virulence of C. perfringens gas gangrene in a mouse model. For this purpose, wild-type C. perfringens type A strain ATCC 3624, an agrB-null mutant and a complemented strain were used. In addition, the possible inhibitory effects of the 6-R peptide on C. perfringens virulence in this gas gangrene model was tested to further evaluate if the Agr-like QS system is important for gas gangrene.

### Results

1. Construction and Genotypic Characterization of an ATCC 3624 agrB-Null Mutant and Complementing Strain.

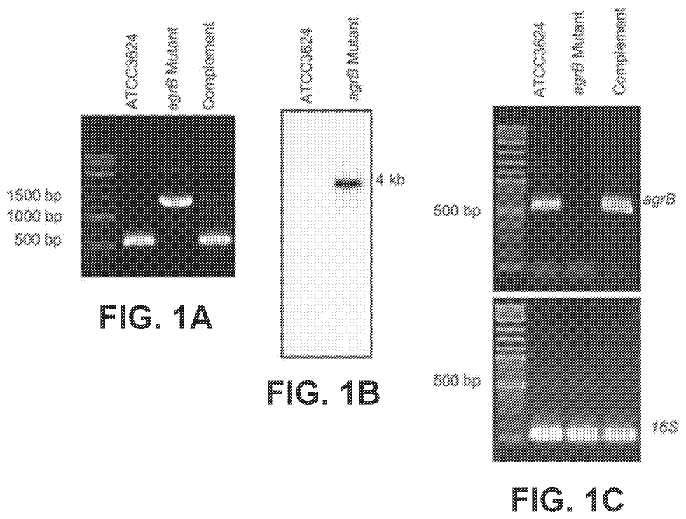

The present study constructed an agrB-null mutant of type A strain ATCC 3624 in order to begin evaluating whether the Agr-like quorum-sensing system regulates the ability of type A strains to produce toxins and cause gas gangrene in a mouse model. For this purpose, the Clostridium-modified TargeTron-mediated insertional mutagenesis method was used to construct an ATCC 3624 agrB-null mutant, as demonstrated by PCR using primers that flank the intron insertion site and are specific for internal agrB open reading frame (ORF) sequences (FIG. 1A). With template DNA from wild-type ATCC 3624, these internal PCR primers specifically amplified a PCR product of 536 bp. However, the same primers amplified a PCR product of about 1.5 kb using DNA from the putative agrB mutant, which is consistent with the insertion of a 900-bp intron into the agrB ORF. The intron delivery plasmid was then cured by sub-culturing for 10 days in the absence of antibiotic selection.

To confirm that only a single intron insertion was present in the putative mutant, DNA was isolated from this strain and subjected to Southern blot analysis using an intron-specific probe (FIG. 1B). The intron-specific probe did not hybridize with wild-type DNA on this Southern blot, as expected. In contrast, this Southern blot experiment revealed that DNA from the agrB-null mutant strain contained a single intron insertion.

A complementing strain was then prepared using a plasmid where the agr operon was cloned into the C. perfringens/Escherichia coli shuttle plasmid pJIR750. After this plasmid was transformed into the ATCC 3624 agrB-null mutant by electroporation, PCR confirmed the presence of the wild-type agrB ORF in the complementing strain. Note that, while FIG. 1A shows the presence of the intron-disrupted agrB gene in the mutant used to prepare the complementing strain, this larger PCR product was not amplified from the complementing strain. This phenomenon has been observed many times previously with complementation of mutants created by intron-mediated insertional mutagenesis. It is due to the primers amplifying products from both the wild-type and intron-disrupted agrB genes in the complementing strain but because of its much smaller size, the PCR product from the wild-type agrB gene is created more rapidly and greatly increases in relative abundance after each PCR round. An RT-PCR assay was then used (FIG. 1C) to assess agrB expression by 5-h TY broth cultures of wild type, the agrB-null mutant and the complementing strain since an AgrB antibody is not available. This reverse transcription-PCR (RT-PCR) analysis confirmed that wild-type ATCC 3624 expresses agrB transcripts. However, no agrB transcription was detectable for the mutant. The complementing strain showed restored agrB transcription with expression levels similar to those of the wild-type strain. For all three ATCC 3624-related strains, the 16S RNA expression levels were similar.

2. Comparison of Growth Rates and Toxin Production by Wild-Type ATCC 3624 Versus an ATCC 3624 agrB-Null Mutant or Complementing Strain in TY Medium.

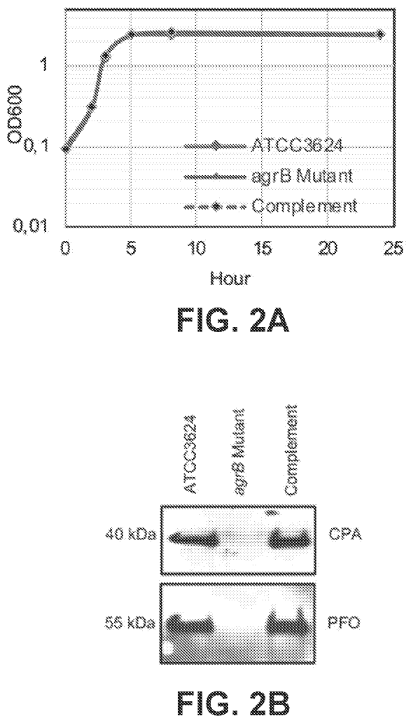

When growth curve analyses were performed (FIG. 2A), wild-type ATCC 3624, its agrB-null mutant, and the complementing strain all grew similarly in TY medium at 37° C. Since type A strains commonly produce CPA and PFO, Western blot studies were performed to compare CPA and PFO production by ATCC 3624, the agrB-null mutant and the complementing strain after overnight (about 16 h) growth in TY medium at 37° C. The results (FIG. 2B) confirmed CPA and PFO production by wild-type ATCC 3624. However, there was no detectable CPA or PFO production by the agrB-null mutant, even after overnight culture. The agrB complementing strain made similar amounts of CPA and PFO as the wild-type parent. These results indicate that, as reported previously for type A strain 13, CPA and PFO production are strongly upregulated by the agr locus in type A strain ATCC 3624.

3. Virulence of Wild-Type C. perfringens Type A Strain ATCC 3624 and its Derivatives in a Mouse Model of Gas Gangrene.

The present study next tested the pathogenicity of C. perfringens type A strain ATCC 3624 and its derivatives in a mouse model of clostridial myonecrosis. For this analysis, ˜10⁶ CFU of wild-type ATCC 3624, the agrB-null mutant and complementing strain were each inoculated intramuscularly into the left hind leg of eight male or female BALB/c mice (weighing 20 to 25 g) per group. A fourth group of eight mice was injected intramuscularly with sterile Dulbecco phosphate-buffered saline (DPBS; control). After 4 h of incubation, the mice were euthanized and examined for gross pathology, and samples were collected for microscopic evaluation and C. perfringens immunohistochemistry (IHC).

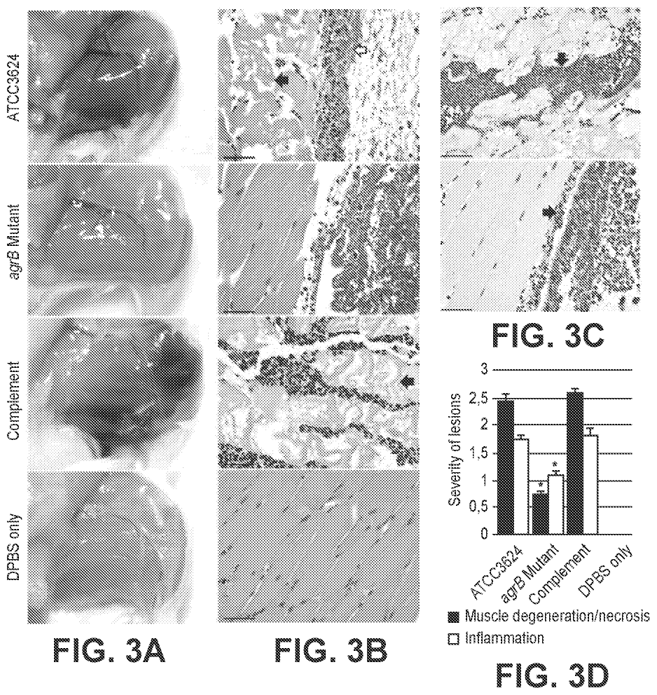

Grossly, wild-type ATCC 3624 and the complementing strain induced severe changes in challenged mice; these changes were characterized by swelling and dark-red discoloration (hemorrhage) of the affected skeletal muscle. In contrast, a similar challenge with the agrB-null mutant induced no gross changes in mouse skeletal muscle (FIG. 3A). The negative control (an injection of DPBS buffer) also failed to induce any gross changes in skeletal muscle.

Microscopically, wild-type ATCC 3624 induced severe histological changes, characterized by muscle degeneration, necrosis, and inflammation with myriad intralesional rods. Leukostasis was prominent within multiple blood vessels (FIG. 3B). Similar challenge with the agrB mutant produced muscle degeneration, necrosis and inflammation with myriad intralesional rods that were significantly less severe than those observed in mice inoculated with the wild-type strain. The severity of these changes reverted to wild-type levels in animals receiving the complementing strain. Mice receiving DPBS only showed no changes in any of the evaluated parameters (FIGS. 3B and 3D). Association of C. perfringens with the microscopic lesions in mice inoculated with wild-type ATCC 3624, the agrB mutant (FIG. 3C), or complementing strain was confirmed by IHC using an indirect immunoperoxidase technique.

4. Recovery of Viable C. perfringens from Challenged Skeletal Muscle.

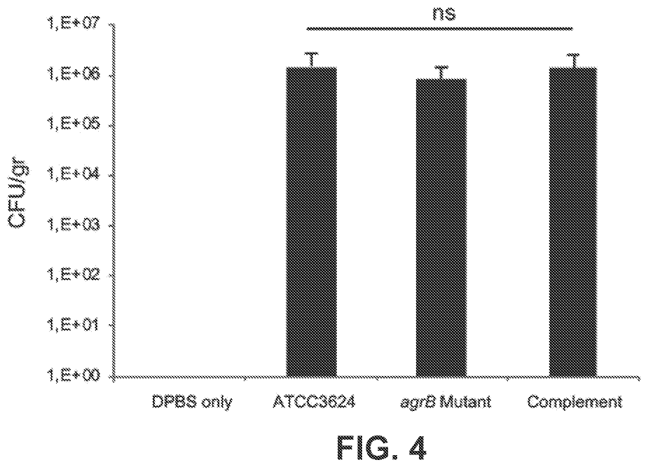

The IHC results indicated that the amounts of total (live and dead) C. perfringens in muscle was similar after challenge with ATCC 3624 or its derivatives (FIG. 3C). To assess whether the numbers of viable C. perfringens present in these tissues were similar, skeletal muscle was aseptically collected from all mice and then plated on C. perfringens selective agar to process for CFU calculations. Similar numbers (˜10⁶) of viable C. perfringens were recovered 4 h after challenge from all mice receiving wild-type ATCC 3624, its agrB-null mutant, or the complementing strain (FIG. 4). Fifteen randomly selected colonies were screened by PCR for the presence of the cpa gene to confirm their identity as C. perfringens. All colonies tested positive (data not shown). No C. perfringens were isolated from mice injected with DPBS buffer alone (controls).

### 5. Effects of the 6-R Synthetic Signaling Peptide on In Vitro CPA or PFO Production by ATCC 3624.

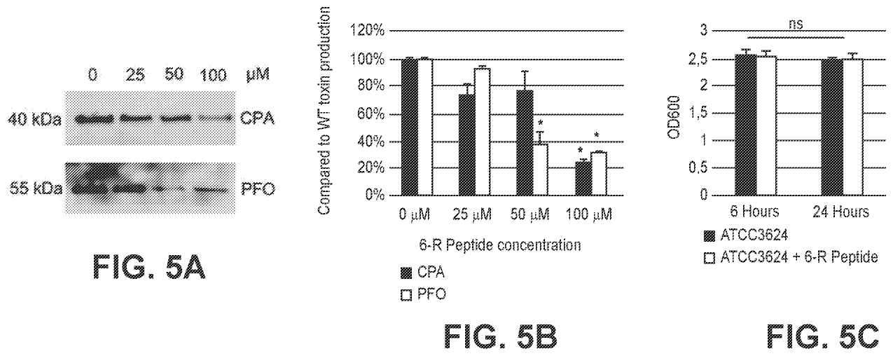

The 6-R peptide is a thiolactone ring consisting of the likely natural SP of C. perfringens plus an additional amino acid that inhibits, by an unidentified mechanism, the Agr-like QS system of several type B and type C wild-type C. perfringens strains, resulting in reduced CPB toxin production by those type B and C strains. Without being bound by theory, there is evidence to suggest that the structures of certain AIPs previously believed to exist as thiolactones may be cysteine-containing homodetic peptides in their functional state, however. Therefore, the present study evaluated whether the 6-R peptide can also inhibit CPA or PFO production by type A strain ATCC 3624. For this analysis, three different concentrations of the 6-R peptide were added to TY broth inoculated with wild-type ATCC 3624, followed by CPA or PFO Western blot analysis of CPA or PFO levels in 5-h culture supernatants of those cultures. The results showed (FIG. 5A) that, at a 100 μM concentration, the 6-R peptide caused an ˜75% reduction in both CPA and PFO production. For PFO production, even a 50 μM concentration of the 6-R peptide had significant inhibitory effects (FIG. 5B). This reduction in toxin production was not due to the 6-R peptide affecting bacterial growth (FIG. 5C).

6. Effects of the 6-R Synthetic Signaling Peptide on C. perfringens Type A-Induced Gas Gangrene In Vivo.

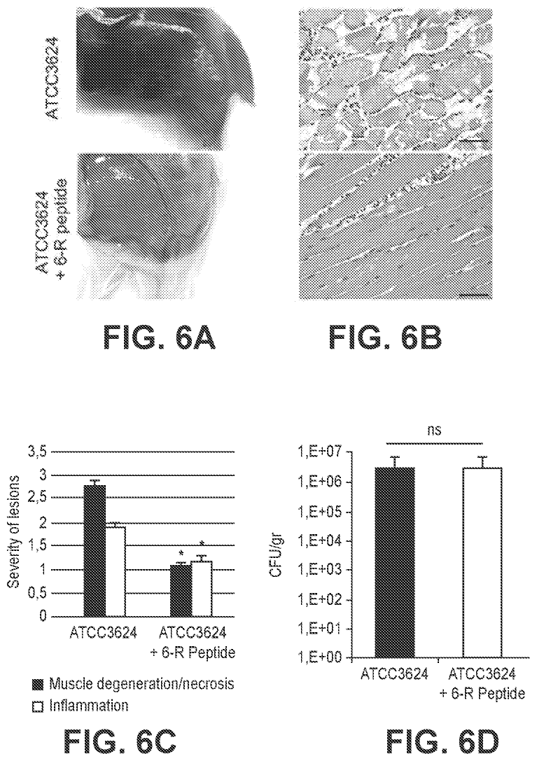

Since the 6-R peptide was able to reduce in vitro production of both CPA and PFO, the present study used this inhibitory peptide to further evaluate involvement of the Agr-like QS system in gas gangrene by testing whether 6-R affects virulence in the mouse model of gas gangrene induced by C. perfringens type A infection. For this experiment, ˜10⁶ CFU of wild-type ATCC 3624 was incubated for 4 h with a 100 μM concentration of the 6-R peptide in dimethyl sulfoxide (DMSO) or an equal concentration of DMSO (control) and then injected into the leg muscles of mice. After 4 h, muscle degeneration/necrosis and inflammation were significantly less severe in animals receiving the 6-R peptide (100 μM) than in mice receiving wild-type ATCC 3624 alone (FIG. 6A-C). Similar numbers (˜10⁶) of viable C. perfringens were recovered 4 h after challenge from all mice receiving wild-type ATCC 3624 or this strain plus 6-R peptide (100 μM) (FIG. 6D). Fifteen randomly selected colonies were screened by PCR for the presence of the cpa gene to confirm their identity as C. perfringens. All colonies tested positive (data not shown). No bacteria were isolated from mice injected with DPBS alone (controls).

### Discussion

The present study establishes an important role for the Agr-like QS system in the pathogenesis of gas gangrene induced by C. perfringens type A in a mouse model. Similar QS communication systems are present across many pathogenic firmicute species where they commonly function as regulators for concerted population behaviors, including biofilm formation, sporulation, and virulence. It has been hypothesized that the coordinated production of extracellular virulence factors when a sufficient population density is reached can induce considerable damage to the host, reducing, at the same time, the use of metabolic resources.

In appropriate C. perfringens types, the Agr-like QS system regulates in vitro production of most extracellular toxins. Involvement of the Agr-like QS in C. perfringens infections originating in the intestine was previously demonstrated for type C and type G strains. In the type C study, an agrB-null mutant of type C strain CN3685 showed reduced enteropathogenicity in a rabbit intestinal loop model and less lethality in a mouse enterotoxemia model; both effects were reversible by complementation. Furthermore, attenuation of this agrB mutant was attributed to reduced production of CPB. Similarly, the virulence of an agrB-null mutant of type G strain CP1 was attenuated in a chicken necrotic enteritis model in vivo and pathogenicity was restored by complementation. Even though NetB production was not quantified in the referred to in vivo study, it is likely that a similar event described for CPB may be responsible for such reduction in pathogenicity in the chicken model since NetB production is controlled by the Agr-like QS and NetB is essential for type G strain-induced chicken necrotic enteritis.

Consistent with previous observations using type A strain 13, inactivation of the agrB gene in ATCC 3624 also produced a significant in vitro decrease in cpa and pfoA expression at the transcriptional level and production of these protein toxins at the translational level. This reduced toxin expression is likely to be responsible for the significant decrease in the severity of macro- and microscopic lesions induced in the skeletal muscle of mice challenged with the agrB mutant since CPA and PFO play central roles in the development of gas gangrene. There was a significant reduction in inflammatory cells at the site of injury in mice receiving the ATCC 3624 agrB-null mutant, but many cells of this C. perfringens strain were still visible. This reduced presence of inflammatory cells is consistent with previous observations, in which C. perfringens type A strains deficient in CPA or PFO production induced significantly reduced leukocyte aggregation in mice.

To test whether the reduced virulence of the agrB-null mutant in the gas gangrene model was due to a decreased ability of this mutant to survive and reproduce in vivo, skeletal muscle from challenged mice was aseptically collected by the time of euthanasia for C. perfringens counting. The number of viable C. perfringens recovered from muscles 4 h after infection was approximately the same as the challenge dose, indicating that C. perfringens does not significantly grow in this infection model, in agreement with results of another recent study. Importantly, no significant differences were observed in the numbers of viable C. perfringens recovered after challenge with the wild-type, mutant, or complemented strains. These results strongly suggest that the reduced virulence of the ATCC 3624 agrB-null mutant in the gas gangrene model was not related to differences in its ability to survive in vivo.

For ethical reasons, this study used a 4-h model based on a pilot study in which this time was sufficient to produce gross and microscopic lesions of gas gangrene, reduce animal suffering and death. This is a modification from previous studies that reported significant clinical sign variations using longer periods models.

A previous study showed that, via an unidentified mechanism, a C. perfringens AgrD sequence-based synthetic peptide named 6-R was able to interfere with CPB production by several C. perfringens type B and C strains in vitro. Why the 6-R peptide blocks or reduces toxin production in only some C. perfringens strains is not understood but it has been speculated that some degree of diversity in C. perfringens SP receptors may exist. The present study determined that the 6-R peptide can also reduce, by ˜75%, the production of CPA and PFO by ATCC 3624 in vitro. Cultures of wild-type ATCC 3624 incubated in the presence of a 100 μM concentration of the 6-R peptide also had reduced ability to produce significant lesions in the skeletal muscle of mice. The reduction of both CPA/PFO production in vitro, as well as the reduced virulence in vivo, was not attributable to the 6-R peptide affecting bacterial growth.

In summary, the present study provides the first evidence that the Agr-like QS system is important for the pathogenesis of gas gangrene in a mouse model of the disease. Given the continuous search for therapies against bacterial pathogens and to avoid the development of antibiotic resistance, interfering with the Agr QS system by using SP analogues like 6-R may represent a potential candidate as a target to prevent some C. perfringens-mediated diseases, including gas gangrene.

### Materials and Methods

a. Bacteria, Media, and Reagents.

C. perfringens type A strain ATCC 3624 was purchased from the American Type Culture Collection (ATCC). The following broth media were used in this study: fluid thioglycolate medium (FTG; Difco Laboratories), TY broth (3% tryptic soy broth [Becton, Dickinson], 1% yeast extract [Becton, Dickinson], and 0.1% sodium thioglycolate [Sigma-Aldrich]), and TGY broth (TY broth supplemented with 2% glucose [Sigma-Aldrich]). The agrB-null mutant and complementing strains were screened using brain heart infusion (BHI) agar (Research Products International) plates containing 15 μg ml-1 chloramphenicol (Sigma-Aldrich). Tryptose-sulfite-cycloserine (TSC) agar plates made of SFP agar base (Becton, Dickinson) with 0.04% d-cycloserine (Sigma-Aldrich) were used for isolation of C. perfringens from challenged skeletal muscle of mice. All other chemical reagents used in this study were purchased from Fisher Scientific, Sigma-Aldrich, or Bio-Rad.

b. Peptide Synthesis.

Based upon results of a previous report, this study used a synthetic 6-mer (6-R) peptide which consists of the sequence CLWFTH (SEQ ID NO: 2) in a thiolactone ring, Synthesis of the 6-R peptide was carried out by the Peptide and Peptoid Synthesis Core Facility Division of the Health Sciences Core Research Facilities (HSCRF) at the University of Pittsburgh. Synthesis was performed using standard FMOC (9-fluorenylmethoxycarbonyl) chemistry cycles on a Liberty CEM microwave synthesizer using Oxyma/DIC [ethyl-(2Z)-2-cyano-2-hydroxyiminoacetate/N,N-diisopropylcarbodiimide] activation in dimethylformamide (DMF). Briefly, FMOC-Thr(tBu), FMOC-His(trt), FMOC-Cys(trt), FMOC-Leu, FMOC-Trp(Boc), and FMOC-Phe were purchased from Peptides International and used for stepwise assembly of the linear sequence on bis(2-sulfanylethyl) amino polystyrene (SEA-PS) resin (0.11 mmol g⁻¹; Millipore Sigma). Cleavage of the 6-R-bis(2-sulfanylethyl) amino intermediate from the SEA solid support was accomplished using a mixture of TFA/TIPS/DMS/H2O/thioanisole (90/2.5/2.5/2.5/2.5) for 2 h at room temperature and then precipitated with diethyl ether, followed by three ether washes. The resulting pellet containing the crude 6-R-bis(2-sulfanylethyl) amino intermediate was allowed to air dry and then dissolved in 0.2 M sodium phosphate/tris(2-carboxyethyl)phosphine (TCEP) at pH 4.0, layered with N2, and incubated overnight at 37° C. with gentle stirring. The resulting crude reaction mixture was directly loaded onto a Waters Prep 4000 series chromatography system and purified on a Phenomenex Gemini (21.2×250 mm) 10-μm C18 column using standard acetonitrile/0.1% TFA gradient conditions. Final analytical determination of peptide purity for the cyclic 6-R-thioester was performed on a Waters Alliance chromatography system using a Phenomenex Gemini (4.6×250 mm) 5-μm C18 column along with standard acetonitrile/0.1% TFA gradient conditions. Matrix-assisted laser desorption ionization-time-of-flight analysis on an Applied Biosystems Voyager workstation was used for final confirmation of the expected target mass of each peptide. The purity of the final peptide was >95%. The purified synthetic peptide was resuspended in DMSO (Fisher Scientific) at 50 mM for use and then stored in a −80° C. freezer for no longer than 2 weeks. In experiments, the final concentrations of 6-R peptide ranged from 25 to 100 μM, as specified.

c. Plasmids and Primers.

An agrB knockout plasmid named pJIR750agrBNi was constructed to prepare an ATCC 3624 agrB-null mutant using Clostridium-modified group II TargeTron Technology. The intron on this plasmid was sense orientation targeted to insert into the agrB ORF between nucleotides 342 and 343. The primers used for intron targeting the agrB gene were as follows:

agrB-342|343s-IBS,
(SEQ ID NO: 3)
5′-AAAAAAGCTTATAATTATCCTTAGTGTTCATTGGAGTGCGCCCAGA
TAGGGTG-3′;
agrB-342|343s-EBS1d,
(SEQ ID NO: 4)
5′-CAGATTGTACAAATGTGGTGATAACAGATAAGTCATTGGAATTAAC
TTACCTTTCTTTGT-3′;
and
agrB-342|343s-EBS2,
(SEQ ID NO: 5)
5′-TGAACGCAAGTTTCTAATTTCGATTAACACTCGATAGAGGAAAGTG
TCT-3′.

The 350-bp intron PCR product was then inserted into pJIR750ai between the HindIII and BsrGI enzyme (New England Biolabs) sites to construct the pJIR750agrBNi vector. The screening primers used to verify the agrB-null mutant were NagrBKOF (5′-TGGAACTTATGCTCTAATACAAACA-3′) (SEQ ID NO: 6) and NagrBKOR (5′-AATCTATAGTTTTTAACAATATATTT-3′) (SEQ ID NO: 7). The same primer pair was also used for RT-PCR analysis of agrB gene expression. 16S RNA was used as a control housekeeping gene for RT-PCR. In this study, all primers were synthesized by Integrated DNA Technologies.

The agrB complementation vector (CPJVp3) was constructed as described previously. This plasmid was electroporated into the ATCC 3624 agrB-null mutant, and transformants were selected on BHI agar containing 15 μg ml⁻¹ of chloramphenicol. The mutant and complementing strains were further confirmed and characterized by PCR, RT-PCR, and Southern blot analyses, as described below.

d. C. perfringens DNA Isolation, PCR, and Intron Southern Blot Analyses.

A MasterPure Gram-positive DNA purification kit was used for DNA extraction from all C. perfringens strains according to the manufacturer's instructions (Epicenter). PCR for the agrB gene was performed using the NagrBOF and NagrBKOR primers. For the wild-type strain the PCR product amplified using these primers was 536 bp, while for the agrB-null mutant strain the same primer pair amplified a PCR product of about 1,400 bp due to the insertion of a 900-bp intron.

For Southern blotting, aliquots (3 μg each) of wild-type or agrB-null mutant DNA samples were first digested overnight with EcoRI at 37° C. according to the manufacturer's instructions (New England Biolabs). The overnight-digested DNA samples were then electrophoresed on a 1% agarose gel, followed by transfer onto a positively charged nylon membrane (Roche) for hybridization with an intron-specific probe (46). The intron-specific probe was prepared using the PCR DIG probe synthesis kit (Roche) and the intron primers (IBS and EBS2). After hybridization, Southern blots were developed using reagents from a DIG DNA labeling and detection kit (Roche), according to the manufacturer's instructions.

e. C. perfringens RNA Isolation and RT-PCR Analyses.

The wild-type parent ATCC 3624 and derivatives (the agrB-null mutant or complementing strain) were grown in TY medium for 5 h at 37° C. Cultures were then pelleted and RNA was extracted using saturated phenol and purified by TRIzol and chloroform (Life Technology and Sigma), as previously described. After the absence of DNA was confirmed by subjecting samples to PCR without reverse transcriptase, RNA was quantified by measuring the sample absorbance at 260 nm. An aliquot of 1 μl of purified RNA (100 ng) was then used in a one-step RT-PCR containing 10 μl of 2×Taq master mix (New England Biolabs), 4 U of avian myeloblastosis virus reverse transcriptase (Promega), and agrB gene primers (described earlier), with ddH₂O added to reach a 20-μl total volume. Similarly, 16S RNA RT-PCR was performed as a loading control. Reaction mixtures were incubated for 45 min at 45° C. to allow cDNA synthesis, and then regular PCR cycling was performed using the following conditions: (i) 95° C. for 2 min; (ii) 30 cycles of 95° C. for 15s, 50° C. for 30 s, and 68° C. for 30 s; and (iii) a final extension of 68° C. for 5 min.

f. Measurement of C. perfringens Growth In Vitro.

For analysis of C. perfringens in vitro vegetative growth, a 0.2-ml aliquot from overnight FTG cultures of the wild type, agrB-null mutant, or complementing strain was inoculated into 10 ml of TY medium. The cultures were then incubated at 37° C.; at various culture times (0, 1, 3, 5, 8, and 24 h) thereafter, a 1-ml aliquot of culture was removed to measure the optical density at 600 nm (OD₆₀₀) by using a Bio-Rad Smart spectrophotometer.

g. Western Blot Analyses of CPA and PFO Production.

TY cultures of the wild-type, agrB mutant, or complementing strain were adjusted to equal optical densities, and 30-μl portions of supernatants from those cultures were then mixed with 5×SDS-PAGE loading buffer and boiled for 5 min. Agr locus controls the expression of a number of secreted proteins, so loading equal amounts of protein in these experiments is not applicable for comparing the relative production of PFO or CPA by wild-type, the agrB mutant, or complementing strain. Instead, the OD₆₀₀ of each culture was adjusted to equivalence (since Agr does not affect growth) and then loaded the same volume of supernatant from that culture. Portions (30 μl) of each sample were then electrophoresed on a 10% acrylamide SDS gel, and the separated proteins were transferred onto a nitrocellulose membrane. The membrane was blocked with TBS-Tween 20 (0.05% [vol/vol]) and nonfat dry milk (5% [wt/vol]) for 1h at room temperature, followed by probing with a 1:1,000 dilution of rabbit polyclonal PFO antibody or a 1:250 dilution of mouse monoclonal CPA antibody overnight at 4° C. Finally, bound antibody was detected with a horseradish peroxidase-conjugated secondary anti-rabbit or anti-mouse antibody (Sigma-Aldrich) and the addition of SuperSignal West Pico chemiluminescence substrate (Fisher Scientific).

h. Inhibition of Toxin Production by the 6-R Peptide.

Wild-type ATCC 3624 was cultured overnight at 37° C. in FTG before reculture in fresh TY broth overnight at 37° C. After that second overnight growth, a 15-μl aliquot of the TY culture was inoculated into 1 ml of TY with or without the specified concentration of 6-R peptide. The culture without 6-R peptide received 2 μl of DMSO as a control since this amount of DMSO was present in cultures receiving the 6-R peptide. After 5 h of culture at 37° C., the supernatants were collected and used for Western blot detection of CPA or PFO production, as described above. ImageJ analysis was performed on three separate Western blots to determine the fold changes of CPA and PFO in the supernatants.

i. Mouse Model of Gas Gangrene.

A 40-μl aliquot from overnight FTG cultures of the wild-type, agrB-null mutant, or complementing strain was inoculated into 1 ml of TY medium, followed by incubation at 37° C. for 5 h. These cultures were then washed in sterile DPBS. An aliquot of 50 μl, equivalent to ˜106 CFU, was injected intramuscularly in the thighs of eight BALB/c mice (20 to 25 g) per group of treatment. Another group of mice was challenged with 50 μl of sterile DPBS (control). The animals were euthanized by cervical dislocation 4 h after infection, and samples of skeletal muscle were collected. All experiments involving mice were reviewed and approved by the University of California, Davis, Institutional Animal Care and Use Committee (protocol 20513).

j. Histopathology.

Sections of challenged skeletal muscle were fixed by immersion in 10% buffered formalin (pH 7.2), for 24 to 72 h. Sections (4 μm thick) were then prepared routinely and stained with hematoxylin and eosin. The sections were examined microscopically by a pathologist in a blinded fashion. A semiquantitative score of lesion severity was assigned to each section using an ordinal scale from 0 (no lesions observed) to 4 (most severe). The following scoring system was used, based on examination of 5, 200× microscopic fields: 0, no lesions; 1, changes observed in 0 to 25% of the area examined; 2, changes observed in 25 to 50% of the area examined; 3, changes observed in 50 to 75% of the area examined; and 4, changes observed in 75 to 100% of the area examined. The following criteria were considered in this score: inflammation (edema, hemorrhage, and presence of inflammatory cells) and changes in muscle fibers (loss of striations, loss of cytoplasm, vacuolation, swelling, and hypercontraction bands).

k. C. perfringens Immunohistochemistry.

Sections of skeletal muscle were processed by an indirect immunoperoxidase technique for C. perfringens as previously described using a Dako EnVision kit (Dako, Carpinteria, Calif.) according to the instructions of the manufacturer. The primary antibody was rabbit polyclonal C. perfringens antibody (GenWay Bio, San Diego, Calif.). Samples of skeletal muscle from mice receiving no C. perfringens inoculation were used as negative controls. Additional negative controls consisted of serial tissue sections of the test tissue incubated with normal rabbit serum instead of the specific antibodies. The colon of a goat from which C. perfringens had been isolated was used as a positive control for C. perfringens immunohistochemistry.

l. Recovery of C. perfringens from Challenged Tissues.

Skeletal muscle was collected aseptically. Tissues were weighed, macerated, and resuspended in DPBS. Serial dilutions (10⁴ to 10⁻⁸) in DPBS were plated on selective TSC agar plates for C. perfringens and anaerobically incubated overnight at 37° C. After 24 h, black colonies, indicative of C. perfringens, were counted to calculate the number of CFU per gram of muscle. A representative number (n=15) of those colonies was screened by PCR for the cpa gene to confirm that they were C. perfringens. For this, the primers CPAF (5′-GCTAATGTTACTGCCGTTGA-3′) (SEQ ID NO: 8) and CPAR (5′-CCTCTGATACATCGTGTAAG-3′) (SEQ ID NO: 9), which amplify a PCR product of about 324 bp, were used. The PCR program was as follows: initial denaturation at 94° C. for 5 min; 30 cycles at 94° C. for 45 s, 56° C. for 45 s, and 72° C. for 60 s; and final extension at 72° C. for 7 min.

m. Inhibition of Gas Gangrene by the 6-R Peptide in a Mouse Model.

Aliquots (40 μl) of an overnight FTG cultures of wild-type, agrB-null mutant, or complementing strains were inoculated into 1 ml of TY medium containing 2 μl of the 6-R peptide (100 μM) in DMSO or an equal concentration of DMSO alone and then incubated at 37° C. for 5 h. The cultures were then washed in sterile DPBS, and 1 μl of the 6-R peptide (100 μM) in DMSO or an equal concentration of DMSO alone was again added. An aliquot of 50 μl—equivalent to ˜10⁶ CFU—was injected intramuscularly in the left thighs of eight BALB/c mice (20 to 25 g) per treatment group. Another group of mice was injected with 50 μl of sterile DPBS (control). The animals were euthanized 4h after infection, and samples of skeletal muscle were collected.

n. Statistical Analyses.

All statistical analyses were performed using R (v3.3.1). Histopathological scores were compared by using a nonparametric Kruskal-Wallis test, followed by the Dunn test as post hoc analysis. For CPA and PFO production in culture supernatants, one-way analysis of variance was applied followed by a post hoc analysis using Tukey's multiple-comparison test. Bacterial counts were compared by negative binomial regression analysis. Differences were considered significant when the P value was <0.05.

### Example II. Evidence that VirS is a Receptor for the Signaling Peptide of the Clostridium perfringens Agr-Like Quorum Sensing System

C. perfringens beta toxin (CPB) is essential for the virulence of type C strains, a common cause of fatal necrotizing enteritis and enterotoxemia in humans and domestic animals. Production of CPB, as well as several other C. perfringens toxins, is positively regulated by both the Agr-like quorum sensing (QS) system and VirS/R two-component regulatory system. This study presents evidence that the VirS membrane sensor protein is a receptor for the AgrD-derived signal peptide (SP) and that the 2nd extracellular loop of VirS is important for SP binding. Understanding interactions between SP and VirS improves the understanding of C. perfringens pathogenicity and may provide insights for designing novel strategies to reduce C. perfringens toxin production during infections.

Since both the Agr-like QS system and VirR/S two-component regulatory system of C. perfringens positively regulate production of several toxins, including beta toxin (CPB), it has been hypothesized the VirS membrane sensor protein is an Agr-like QS signaling peptide (SP) receptor. To begin evaluating whether VirS is an SP receptor, this study sequenced the virS gene in C. perfringens strains CN3685 and CN1795 because it was reported that agrB mutants of both strains increase CPB production in response to the pentapeptide 5R, likely the natural SP, but only the CN3685 agrB mutant responds to 8R, which is 5R plus a 3 amino acid tail. This sequencing identified differences between the predicted VirS second extracellular loop (ECL2) of CN3685 versus CN1795. To explore if those ECL2 differences explain strain-related variations in SP sensitivity and support VirS as an SP receptor, virS/agrB double null mutants of each strain were complemented to swap which VirS protein they produce. CPB Western blotting showed this complementation changed the natural responsiveness of each strain to 8R. A pull-down experiment using biotin-5R demonstrated VirS can bind SP. To further support VirS:SP binding, and identify a VirS binding site for SP, a 14-mer peptide was synthesized corresponding to VirS ECL2. This ECL2 peptide inhibited 5R signaling to agrB mutant and wild type strains. This inhibition was specific since a single N to D substitution in the ECL2 peptide abrogated these effects. Collectively, these results support VirS as an important SP receptor and may assist development of therapeutics.

### Introduction

The Gram-positive, anaerobic bacterium Clostridium perfringens is a major cause of histotoxic and intestinal infections in humans and other animals. C. perfringens infections such as gas gangrene, enteritis and enterotoxemia are mediated, in large part, by the ability of this bacterium to produce more than 20 different toxins. However, individual strains produce only subsets of this toxin repertoire, allowing for a classification system based on carriage of genes encoding major toxins, i.e., alpha (CPA), beta (CPB), epsilon (ETX), iota (ITX), enterotoxin (CPE) and NetB toxin. This scheme assigns C. perfringens isolates to one of seven types (A to G).

Of importance for the current study, type B isolates carry genes encoding CPA, CPB and ETX toxins, while type C isolates only carry genes encoding CPA and CPB toxins. Each C. perfringens type is associated with particular diseases, e.g., type B and C strains cause necrotizing enteritis and enterotoxemia. Toxin production by pathogenic bacteria is often regulated by quorum sensing (QS) systems and two-component regulatory systems (TCRS), which can work in tandem. Consistent with that general theme, C. perfringens toxin production is frequently regulated by both TCRS and QS systems. The two most common regulatory systems for controlling toxin production in C. perfringens are the VirS/VirR TCRS and the accessory gene like regulator (Agr) QS system.

The VirS/VirR TCRS includes the membrane sensor histidine kinase VirS and the response regulator VirR. This system, encoded by the virR/S operon, becomes activated by a signal that induces autophosphorylation of VirS, which is followed by phosphotransfer to VirR. Phosphorylated VirR can directly regulate expression of some C. perfringens toxin genes, such as the netB gene encoding NetB or the pfoA gene encoding the perfringolysin O (PFO, by binding to VirR boxes located upstream of the promoters for those genes. Alternatively, phosphorylated VirR indirectly regulates other C. perfringens toxin genes (such as CPA) by inducing expression of a small regulatory RNA named VR-RNA (18). The VirS/VirR TCRS can be important for C. perfringens virulence. For example, this TCRS was shown to be essential for type C strain CN3685 to cause intestinal pathology in rabbit small intestinal loops or enterotoxemic lethality in mice. Consistent with this pathogenic role, the VirS/R TCRS was found to regulate CPB production in vivo, which is important since CPB plays a critical role when type C strains cause necrotic enteritis or enterotoxemia. The Agr QS system was first discovered in Staphylococcus aureus, where it controls expression of genes encoding many toxins and degradative enzymes in a quorum-sensing manner. In S. aureus, an autoinducing signaling peptide (named AIP) is encoded by the agrD gene and modified and secreted by the AgrB membrane protein. In the same S. aureus operon encoding AgrB/D are genes encoding the AgrC/A TCRS. In this system, AgrC is a sensor receptor protein for the AIP and acts by phosphorylating the transcriptional regulator AgrA to induce expression of a gene, located upstream of the agr operon, that encodes a small regulatory RNA named RNAIII. RNAIII then regulates toxin production.

Interestingly, while C. perfringens has an operon encoding AgrB and AgrD, genes encoding AgrC/A or RNAIII are not present in the C. perfringens genome. Nonetheless, the C. perfringens Agr-like QS system regulates the production of several toxins, such as PFO, CPA, CPB and NetB. Consequently, the Agr-like QS system is an important virulence regulator. For example, this QS system was shown to be necessary for the pathogenicity of both type C and NetB-positive type G strains.

In previous work, C. perfringens type C strain 96 CN3685 and type B strain CN1795 agrB mutants, which carry an intron insertion in their agrB gene and do not express the operon encoding AgrB and AgrD, were shown to vary in their sensitivity to different synthetic C. perfringens signal peptide (SP)-based peptides. For both strains, their agrB mutants respond to 5R, which likely corresponds to the natural 5-mer SP of C. perfringens, while only the CN3685 agrB mutant can respond to 8R, which is SP plus a 3 amino acid tail. Since production of several C. perfringens toxins, including CPB, is positively regulated by both the Agr-like QS system and VirR/S TCRS, it has been suggested that the VirS membrane protein is an SP receptor. However, this correlation has not yet been supported by any direct experimental evidence indicating that the SP can bind to and signal VirS. In this study, several lines of evidence that the SP interacts with and signals VirS and that this process involves SP binding to the main extracellular loop of VirS are provided.

### Results

a. Quantitation of agrD, virS and Cpb Expression by Real-Time qRT-PCR.

Previous studies have reported that, i) CPB toxin production is regulated by both the Agr-like QS system and the VirR/S TCRS, and ii) CPB is generally produced in the early stationary phase when C. perfringens populations had achieved high cell density. Therefore, the present example first examined the linkage between cpb transcript levels and agrD or virS expression levels using qRT-PCR.

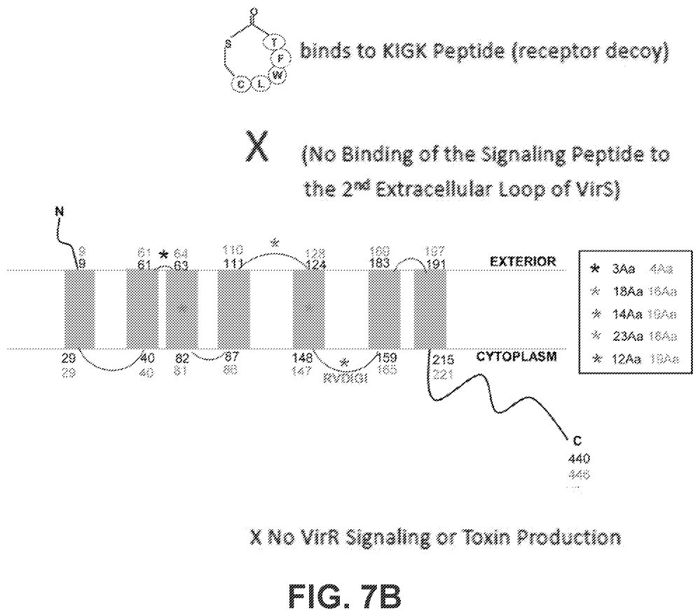

For this analysis, growth curves were first determined (FIG. 8A) for a type B strain (CN1795) and a type C strain (CN3685) cultured in TY medium and production of CPB in those same cultures was also evaluated by Western blotting at various time points (FIG. 8B). The results showed that both strains grew similarly and that their CPB production began 3 h after inoculation (during the logarithmic growth phase). The production of CPB then increased further until 8 h (during stationary phase). Compared to 1 h after inoculation, cpb gene transcript levels had increased by 3 h post-inoculation and then reached maximum levels at 5 h (during early stationary phase) post-inoculation. Levels of the cpb gene transcript were barely detected in 8 h (stationary phase) or overnight (16 h, death phase) cultures (FIG. 8B).

Using the same cDNA samples, agrD and virS transcript levels were also determined by qRT-PCR. The results showed that the agrD transcript level pattern was very similar to the cpb expression pattern (FIG. 8C left panel). However, virS gene transcript levels were maximally expressed 1 h post-inoculation and then decreased thereafter (FIG. 8C, right panel). The patterns of agrD or virS expression were very similar in both C. perfringens strains examined.

b. Sequence Differences Between the virS Gene in CN1795 Versus CN3685.

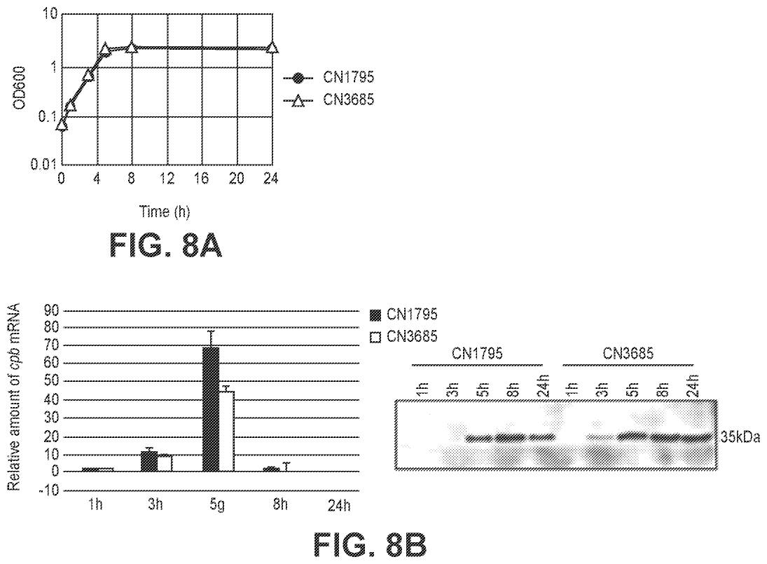

Previous reports described agrB mutants of type B strain CN1795 and type C strain CN3685. These mutants have an intron insertion in their agrB gene that inactivates expression of both the agrB and agrD 133 genes, which are located in the same operon. It is known that the CN1795 and CN3685 agrB mutants respond to SP-based synthetic peptides (FIG. 9A) named 5R (a 5 amino acid thiolactone ring that likely corresponds to the natural C. perfringens SP, but which, without being bound by theory, may be a cysteine-containing homodetic peptide in its functional state as suggested for certain AIPs), but only the CN3685 agrB mutant responds to the 8R peptide, which has the sequence of 5R plus a three amino acid tail. The current study first confirmed those previous observations using newly synthesized 5R and 8R peptides. The results, shown in FIG. 9A, indicated that the CN1795 agrB null mutant only responded to the 5R peptide. In contrast, the CN3685 agrB/D null mutant responded to both the 5R and 8R peptides, although there was stronger signaling (more CPB production) with the 140 5R vs. the 8R peptide, consistent with the previous reports.

The VirS membrane sensor protein of the VirR/S TCRS has been suggested as a potential receptor for the AgrD signaling peptide. As the two agrB null mutant strains respond differently to the 5R or 8R peptides, the VirS protein made by these two strains was compared using virS sequencing. Differences detected between the deduced VirS sequence of these two strains are shown in TABLE 1, and include 3 single amino acid differences at residues 81, 360 and 382 (relative to the CN1795 VirS sequence). However, the most striking difference noted between the virS open reading frame (ORF) of these two strains is that the virS ORF in CN3685 encodes a 6 amino acid insertion at the 152nd amino acid, producing a protein of protein of 446 amino acids compared to the 440 amino acid VirS made by CN1795.

TABLE 1
Sequence differences between the VirS protein of CN1795 vs. CN3685

| | 81st Aa* | 152nd Aa | 360th Aa | 382nd Aa |
|---|---|---|---|---|
| CN1795VirS | R | | S | I |
| CN3685VirS | K | RVDIGI (SEQ ID NO: 14) | N | V |

*Amino acid (Aa) numbers shown refer to the amino acid position in the CN1795 VirS protein.

The transmembrane prediction algorithm (TMHMM) predicts that the VirS protein has 4 extracellular regions that might interact with SP if VirS is an SP receptor (FIG. 9B). Furthermore, the TMHMM program predicts that the sequence differences detected between CN3685 vs. CN1795 impact VirS structure, as shown in FIG. 9B. Of particular interest for this study, these sequence differences are predicted to result in an extracellular loop 2 (ECL2) of 14 amino acids for the CN1795 VirS protein vs. an ECL2 of 19 amino acids for the CN3685 VirS protein (FIG. 9B), blue * in original).

c. Preparation of virS/agrB Double Null Mutant Strains of CN1795 or CN3685 and Swapping of the virR/S Operon Expressed by Those Double Null Mutant Strains.

Given the differences in 8R signaling sensitivity observed between CN1795 and CN3685 (FIG. 9A), and the predicted structural differences in their VirS proteins (FIG. 9B), we inactivated the virS gene in our existing agrB mutants and then complemented those double mutants to swap which VirS protein they expressed in order to test if this swap affects 8R sensitivity, as might be anticipated if VirS is an SP receptor.

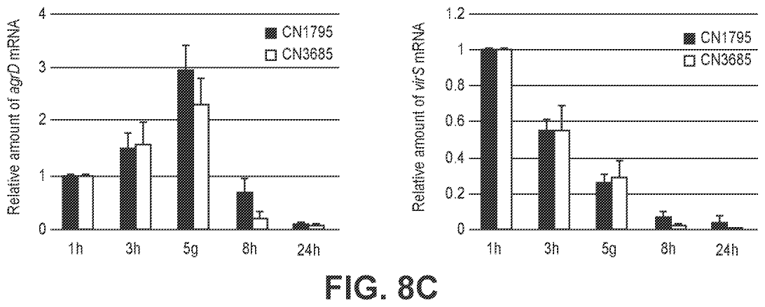

For this purpose, a Clostridium-modified Targetron insertional mutagenesis method was employed to introduce a targeted intron into the virS gene of CN1795agrBKO and CN3685agrBKO to create double virS/agrB null mutants that do not express either the virS gene or the operon encoding agrB/D. The identity of those putative double mutants, named CN1795DKO and CN3685DKO, was first demonstrated by PCR using primers specific for internal agrB ORF or virS ORF sequences. Compared to the PCR products amplified from the agrB or virS genes in wild-type strains, PCR of DNA from the double mutants amplified larger bands from both genes due to insertion of an intron into the wild-type agrB and virS genes (FIG. 10A).

To create complementing strains producing a swapped VirS protein (e.g. CN1795 producing the VirS protein of CN3685), the virR/S operons of CN1795 or CN3685 were separately cloned into the pJIR750 shuttle plasmid. The plasmid carrying the CN1795 virR/S operon was then electroporated into CN3685DKO and the plasmid carrying the CN3685 virR/S operon was electroporated into CN1795DKO, creating (respectively) two complementing strains named CN3685DKOc1795virR/S and CN1795DKOc3685virR/S that now expressed the virR/S operon of the other strain. FIG. 10A PCR results confirmed that, in these two complementing strains, the presence of a wild-type virS gene was restored. Furthermore, an intron-specific Southern blot (FIG. 10B) demonstrated that, while only one intron is present in the single agrB mutant strains, the double mutant strains now carry two intron insertions. RT-PCR results (FIG. 10C) demonstrated that, in the double null mutant strains, neither the agrB nor virS genes were expressed, while the swapped virR/S complementing strains exhibited restored virS gene expression. Western blot results (FIG. 10D) confirmed that CPB, which is regulated by both the Agr-like QS system and VirS/R TCRS, was not expressed in either double null mutant strain, even when the 5R signaling peptide was added.

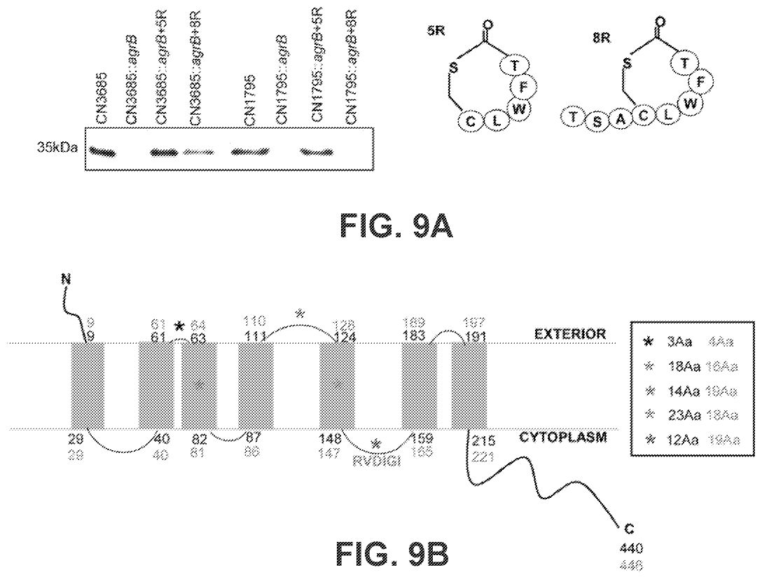

The main purpose of constructing these complementing strains producing a swapped VirS was to test their responsiveness to signaling by peptides 5R or 8R, as assessed by CPB Western blot (FIG. 11A-B). The CN1795 complementing strain expressing CN3685 VirS now responded to both the 5R and 8R peptides, similar to CN3685 agrB KO but unlike CN1795 agrB KO. However, the CN3685 complementing strain expressing CN1795 VirS responded only to the 5R peptide, unlike CN3685 agrB KO but resembling CN1795 agrB KO. While this complementing strain also expressed trace amounts of CPB even without addition of a signaling peptide, likely attributable to VirR overproduction due to complementation with a multicopy plasmid, this CPB production did not increase further when supplied the 8R peptide. We also tested CPB production by this complementing strain when incubated with or without 8R for different time-points (3 h, 5 h, 8 h or overnight), but the presence of 8R did not increase CPB production compared to the absence of 8R at any time point (data not shown).

d. Evidence that a Biotin-Labeled 5R Peptide (B-5R) can Bind to the His₆-Tagged (SEQ ID NO: 43) VirS Protein of CN3685.

Attempts to prepare a VirS antibody for use in a B-5R pull-down experiment were unsuccessful. Therefore, we used a CN3685 strain producing His₆-tagged (SEQ ID NO: 43) VirS and a His₆ (SEQ ID NO: 43) antibody Western blot to determine, in a pulldown assay, whether VirS can physically bind with B-5R, as would be expected if VirS is an SP receptor in C. perfringens.

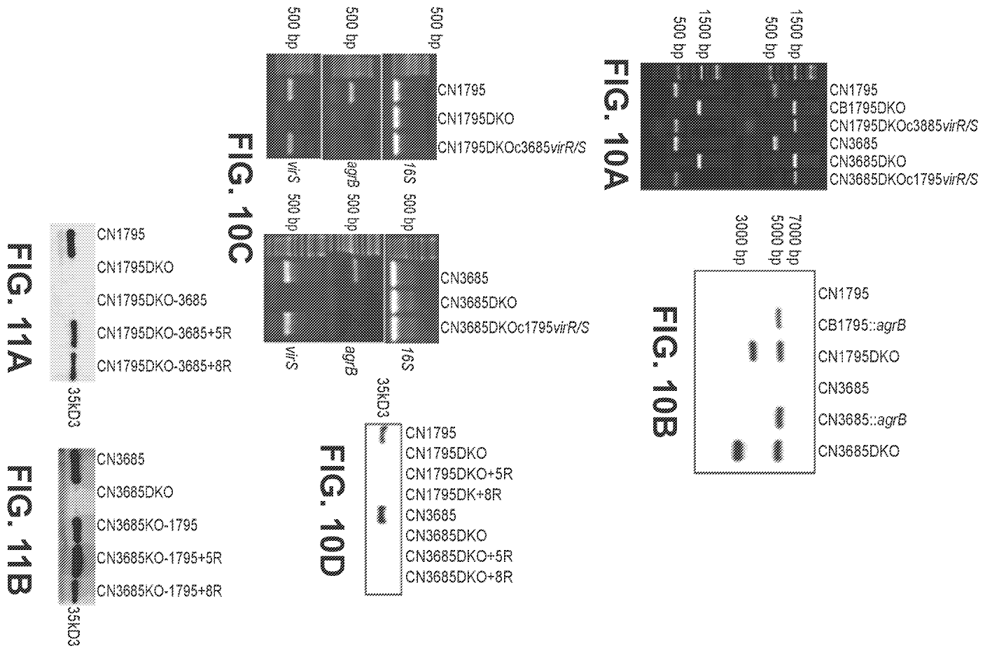

For this purpose, the virS gene in CN3685 was inactivated by insertion of an intron using the Clostridium-modified Targetron insertional mutagenesis method (31). Construction and characterization of this mutant, named CN3685::virS, is shown in FIG. 12. Specifically, PCR analyses (FIG. 12A) demonstrated the insertion of a ˜900 bp intron into the virS gene since the PCR product amplified from this mutant strain DNA was 900 bp larger than the PCR product amplified from the wild-type CN3685 virS gene. An intron-specific Southern blot (FIG. 12B) confirmed the presence of only one intron in this mutant.

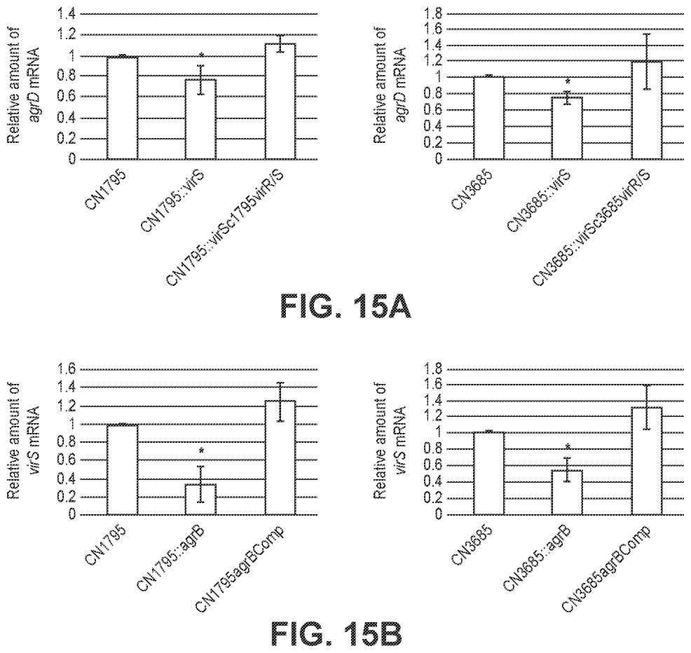

Complementing strains named CN3685::virSc3685virR/S or CN3685::virSc1795virR/S were prepared that produce, respectively, the VirS of either CN3685 or CN1795. As expected, DNA from these complementing strains supported amplification of a PCR product of the same size as the wild-type virS gene (FIG. 12A). RT-PCR (FIG. 12C) analyses detected no virS gene expression by the mutant, while the two virR/S complementing strains showed virS gene expression. Western blot analyses (FIG. 12D) detected no CPB production by the CN3685 virS null mutant strain, while both complementing strains did produce CPB, confirming that their complemented VirS expression enabled functional signaling. At the same time, we similarly prepared a CN1795 null mutant (CN1795::virS) with an intron insertion in virS and a complementing strain producing the CN1795 VirS for studies described later in FIG. 15.

For use in the pull-down experiment, we also transformed the CN3685 virS null mutant to produce VirS protein with a C-terminal His₆ tag (SEQ ID NO: 43), creating CN3685::virSc3685virR/Shis; the only difference between this complementing strain and CN3685::virSc3685/R/S is that a His₆-tag (SEQ ID NO: 43) was added to the VirS C-terminus using a 5′ to 3′ PCR primer. For CN3685::228 virSc3685virR/Shis, CPB Western blotting detected similar CPB production as observed for N3685::virSc3685cvirR/S (data not shown), confirming that the presence of the His₆-tag (SEQ ID NO: 43) did not interfere with VirS function.

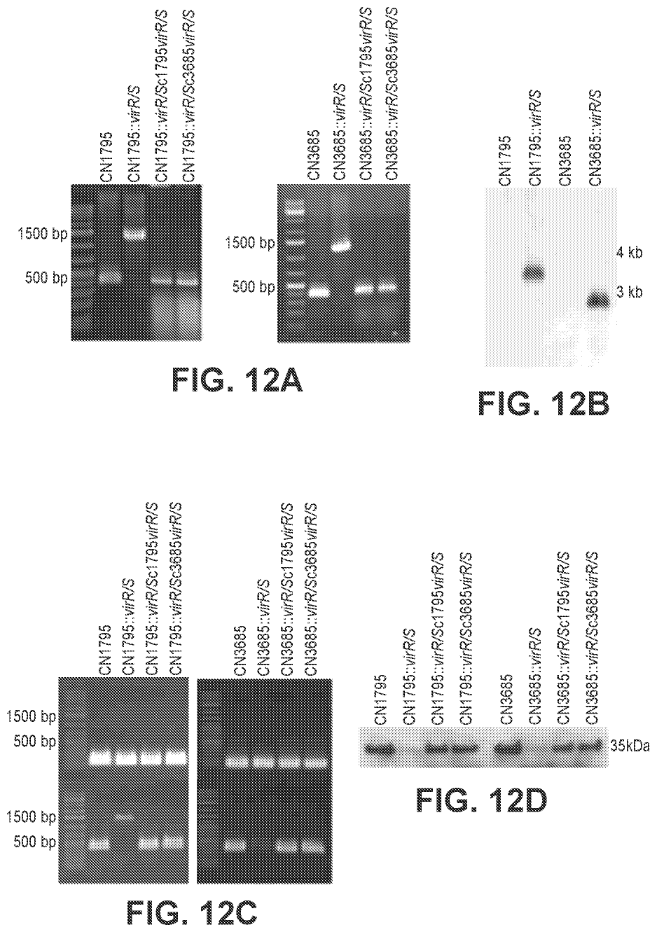

Before using a biotin-labeled 5R peptide (B-5R) in a pull-down experiment to test for its interactions with VirS, it was important to evaluate whether this biotin-modified peptide retains biologic activity, i.e., signaling function. Western blot analysis showed that B-5R induced CPB production by both the CN1795 and CN3685 agrB null mutant strains (FIG. 13A).

With positive results from those control experiments, we then performed a pull-down experiment to evaluate if VirS can physically bind to SP. This involved preincubating streptavidin coated beads with B-5R or 5R before mixing those beads with B-per buffer cell extracts from 4 h TY cultures of CN3685::virSc3685virR/Shis cells (FIG. 13B, lane 1). When those extracts were Western blotted with His₆-specific (SEQ ID NO: 43) antibody prior to incubation with the B-5R or 5R treated beads, a major immunoreactive band was detected that matched the ˜50 kDa size of VirS, although higher molecular mass species were also present, including an ˜100 kDa species that is likely a VirS dimer. After incubation of those extracts with the B-SR-coated streptavidin beads, much of the His₆ (SEQ ID NO: 43) antibody-immunoreactive material, particularly the apparent dimer, remained unbound in the supernatant (lane 2).

As a specificity control, Western blotting with His₆-tag (SEQ ID NO: 43) antibody detected (lane 3) no immunoreactive material in the crude extract of a control complementing strain (CN3685::virSc3685virS/R) that does not produce His₆-tagged (SEQ ID NO: 43) VirS, confirming that the immunoreactive material present in CN3685::virSc3685virR/Shis extracts was attributable to His₆-tagged (SEQ ID NO: 43) VirS. In addition, no immunoreactive material was detected by His₆ (SEQ ID NO: 43) antibody using extracts of CN3685::virSc3685virR/S after pull-down by beads preincubated with either 5R- or B-SR- (lanes 6 and 7). However, an ˜50 kDa immunoreactive species was pulled down from extracts of CN3685::virSc3685virR/Shis using beads preincubated with B-5R, which contains biotin and binds well to streptavidin-coated beads, but not from extracts of those cells preincubated with 5R, which lacks biotin so it does not bind well to streptavidin-coated beads. This Western blot result supports the ability of C. perfringens VirS to physically bind SP.

e. A Synthetic Peptide Corresponding to the Predicted VirS Second Extracellular Loop inhibits SP-induced CPB production.

The ability of B-5R-coated beads to pull-down VirS supports the binding of SP to VirS as a receptor. To confirm binding interactions between 5R and VirS by an independent approach, and to discern a region of VirS involved in SP binding, we used the VirS structural model (FIG. 9B) to identify predicted extracellular regions of the VirS protein that could interact with SP. Since the predicted VirS ECL2 of CN1795 and CN3685 have significant size differences and those strains respond differently to signaling by 8-R (FIG. 9), we hypothesized that this VirS loop is involved in SP binding. To test this hypothesis, we synthesized a 14 amino acid peptide named KIGK (SEQ ID NO: 10) (TABLE 2) that corresponds to the sequence of the predicted ECL2 of CN1795 VirS, as well as a random sequence control peptide with a molecular weight similar to that of KIGK.

TABLE 2
Synthetic peptide sequences

| Peptide | Sequence | MW |
|---|---|---|
| KIGK | NH2-KINSLVNVSELLGK-CO2H (SEQ ID NO: 10) | 1513.80 |
| Control | NH2-AYSSGAPPMPPFFF-CO2H (SEQ ID NO: 11) | 1515 |
| KIGK_D | NH2-KINSLVDVSELLGK-CO2H (SEQ ID NO: 12) | 1514.78 |

Bolded residues show the single different amino acid residue present in KIGK vs. KIGK_D

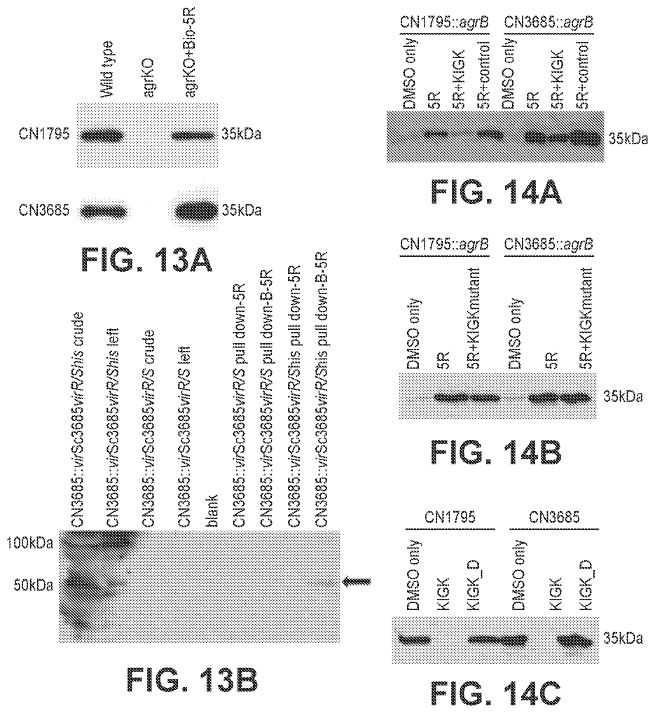

To test if 5R can bind to KIGK, as might be anticipated if Agr-like QS signaling involves SP binding to the predicted ECL2 of VirS, the 5R and KIGK peptides were preincubated together before their addition to cultures of CN1795 or CN3685 agrB null mutant strains. CPB Western blots (FIG. 14A) showed that neither agrB null mutant strains produce CPB in the absence of 5R, as expected from FIG. 9 results. However, consistent with the hypothesis that 5R binds to the 2nd ECL of VirS, preincubation of 5R with KIGK blocked Agr-like QS signaling for the agrB null mutants, i.e., CPB production was significantly reduced. In contrast, similar preincubation of 5R with the control peptide did not block SP signaling to agrB null mutant strains, i.e., strong CPB production was detected. To further confirm the specificity of the binding interaction between 5R and KIGK, a peptide named KIGK_D (SEQ ID NO: 12) was prepared where the N residue located in the middle of the predicted VirS second extracellular loop was switched to D (TABLE 2). Preincubation of 5R with this peptide had no effect on subsequent SP signaling to agrB mutants, i.e., CPB production was not reduced (FIG. 14B).

To further test our hypothesis that SP binding involves the ECL2 of VirS, we applied KIGK (500 μM or 1 mM) to CN1795 and CN3685 to determine if this peptide can also block Agr-like QS signaling by those two wild-type strains. Results (FIG. 14C) showed that the presence of a 1 mM concentration of the KIGK peptide efficiently blocked CPB production by both wild-type strains, while the same dose of the KIGK_D peptide had no effect on CPB production. A 500 μM concentration of KIGK can partially block CPB production by these strains (data not shown).

f. Effects of the KIGK Peptide on C. perfringens Type A-Induced Gas Gangrene In Vivo.

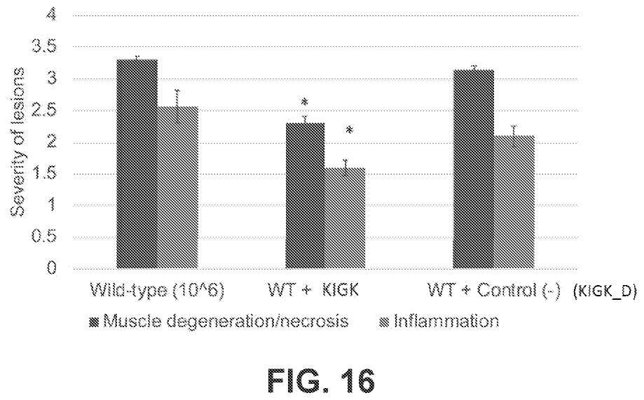

Since KIGK efficiently blocked the in vitro production of CPE, an experiment next tested whether this peptide could reduce the symptoms of gas gangrene caused by C. perfringens in a mouse model. As shown in FIG. 16, the presence of KIGK peptide caused a significant reduction in muscle histologic lesions. In contrast, the negative control KIGK_D peptide did not reduce muscle damage caused by C. perfringens in this model.

g. Cross-Talk Regulation of Expression Between the virR/S Operon and agrB/D Operon.

In S. aureus, it is known that sensing of the AIP causes the receptor AgrC to undergo histidine autophosphorylation and then phosphorylate AgrA, which initiates a positive feedback loop to make more AgrB and AgrD. Since FIGS. 10 to 14 results provided substantial evidence supporting VirS as an SP receptor, further tests were performed to determine whether a positive feedback loop also functions in C. perfringens to help regulate expression of the Agr-like QS system and the VirS/R TCRS. This first involved using virS null mutant strains to study if the presence of VirS, an SP receptor, affects 294 agrD expression. Based upon the kinetics for agrD expression determined in FIG. 8, expression levels of the agrD gene in 5-h TY medium cultures were compared between the two wild-type strains CN1795 and CN3685, as well as cultures of their virS null mutants and their virR/S operon-complemented strains. Results from these qRT-PCR analyses revealed significantly higher agrD transcript levels in the wild-type and its virS/R-complemented strain compared to the virS null mutant strains (FIG. 15A).

To investigate whether SP production affects virS expression, the CN1795 and CN3685 agrB null mutant strains were used. Note that, since agrB and agrD (and two upstream genes) are located within the same operon, the agrD gene is not expressed in null mutant strains with an insertion in their agrB gene. Because virS expression levels peak early, i.e. 1 to 2 hours (FIG. 8), transcript levels of the virS gene were compared in 2-h TY medium cultures for both wild-type strains, their agrB null mutants and those mutants complemented with the agr operon. Those qRT-PCR analyses detected significantly higher virS transcript levels in the wild-type and its agr operon complemented strains compared to the agrB null mutant strains (FIG. 15B).

### Discussion

Producing toxins such as CPB is critical for C. perfringens pathogenicity. Previous studies established that regulation of toxin production, including CPB production, in C. perfringens often involves both the Agr-like QS system and the VirS/R TCRS. However, major gaps have remained in understanding how those systems regulate C. perfringens toxin production, e.g., do these systems work cooperatively or independently? In comparison to the agr operon of the S. aureus Agr system, the C. perfringens agr-like operon encodes a different SP and does not encode a homolog of AgrC, which is the SP receptor in the S. aureus Agr QS system. Instead, since both the Agr-like QS system and VirS/VirR TCRS positively regulate production of several C. perfringens toxins, it has been proposed VirS may be an SP receptor in C. perfringens. However, this important hypothesis had never been directly tested.

Given that CPB production is positively regulated by both the Agr-like QS system and the VirS/R TCRS, the current study used production of this toxin as a read-out for SP signaling. Consequently, initial experiments in this study compared the timing of cpb, agrD and virS expression in C. perfringens. Consistent with previous reports that CPB is most strongly produced during late log-phase growth by type B or C isolates in TGY medium, the example above determined that, in TY cultures of either type B strain CN1796 or type C strain CN3685, cpb transcript levels increased from 1 h until reaching a peak at 5 h, a time corresponding to late log phase-early stationary phase. Some transcription of agrD was observed even during early growth but this expression also peaked at ˜5 h, consistent with previous studies showing that CPB production is controlled by the Agr-like QS system. In contrast, the virS gene was most strongly expressed early during growth, as would be expected if significant amounts of VirS membrane sensor already need to be available when the Agr-like QS maximally signals to increase CPB production.

This example further tested directly whether VirS could be an SP receptor involved in regulating CPB production by C. perfringens type B or C strains. Since previously reported results showed that an agrB mutant of type C strain CN3685 upregulates CPB production in response to both the 5R and 8R synthetic peptides, but an agrB mutant of type B strain CN1795 upregulates CPB production only in response to 5R, if VirS is a major SP receptor then there should be sequence differences between the VirS membrane sensor histidine kinase of these two strains. Sequencing identified several differences between the deduced VirS amino acid sequence of these two strains as shown in TABLE 1. When computer modeling was performed to predict the structural impact of those sequence variations, several structural differences were predicted, including differences in the predicted VirS ECL2. Results of experiments described above support a role for the 2nd ECL loop in SP binding. For example, preincubation of a synthetic peptide corresponding to ECL2 with 5R, the likely natural SP, inhibited signaling to increase CPB production in agrB mutants of CN3685 or CN1795.

This 2nd ECL loop is predicted by computer modeling to include 14 amino acids (extending from amino acids 111-124) in the VirS made by CN1795 but to be 19 amino acids (extending from amino acids 110 to 128) in the VirS made by CN3685. While not wishing to be bound by theory, relative to the VirS made by CN1795, the larger size of its ECL2 may allow the CN3685 VirS to functionally dock and accommodate the larger 8R (as well as the likely natural 5R) for signaling. The results with ECL2 peptides (discussed in further detail below) implicated the Asp residue present at residue 117 in the CN1795 VirS as being important for SP binding. Detailed analysis of this loop should be performed in the future since it represents a potential therapeutic target.

The VirS variant made by CN3685 is unusual. GenBank analysis of deduced VirS sequences in 49 other strains representing many C. perfringens types and origins indicated (data not shown) that only 2 of 49 strains resemble CN3685 in possessing a six amino acid insertion at amino acid 152. This analysis also revealed that the KINSLVNVSELLGK (SEQ ID NO: 10) ECL2 sequence of the CN3685 and CN1795 VirS was reasonably conserved among 49 strains, with only single strains having an N (versus S) at position 4, an S (versus N) at position 7 or an I (versus V) at position 8. More common ECL2 sequence variations in these 49 strains were 16 strains producing an ECL2 with an S (versus a V) at position 6 and 10 strains producing an ECL2 with a D (versus an E) at position 10. Whether those ECL2 variations affect SP signaling should be investigated in the future. ECL2 sequence variations are encompassed by SEQ ID NO: 13, Lys-Ile-Asn-Xaa-Leu-Xaa-Xaa-Xaa-Ser-Xaa-Leu-Leu-Gly-Lys where position 4 is Ser or Asn, position 6 is Val or Ser, position 7 is Asn or Ser, position 8 is Val or Ile, and position 10 is Glu or Asp. Those findings also extend understanding of the VirS structure vs. function relationship.

Consistent with the modeling shown in FIG. 9, previous modeling work had also reported that the VirS sensor histidine kinase likely contains seven transmembrane (TM) domains and a C-terminal tail. That previous study using random mutagenesis did not identify a putative SP binding site. However, it did determine that the predicted TM4 domain of the VirS N-terminal region is important for PFO production, which is regulated by both the VirS/R TCRS and the Agr-like QS system. Since modeling predicts that TM4 is adjacent to the 2nd ECL, the findings described herein may suggest the TM4 domain perturbations that altered PFO production in that previous study affected the 2nd ECL structure and thus altered SP binding and signaling. The previous random mutagenesis study also identified regions of the C-terminal tail of VirS that are important for signaling, including the H255 residue that is an autophosphorylation site and G and N boxes that are involved in ATP binding and catalysis. The current results now add to that previous VirS structure vs function information by implicating the 2nd ECL in SP binding and signaling.

Considerable progress was achieved towards the main goal of the study, i.e., testing the hypothesis that VirS is an SP receptor in C. perfringens. Several lines of evidence were obtained that now directly support VirS as an SP receptor. First, previous observation that both 5R and 8R can signal agrB null mutants of CN3685 while 5R (but not 8R) signals agrB null mutants of CN3718, which suggested that there may be differences between the VirS of CN3685 vs. CN3718 if VirS is a major SP receptor, was exploited. As discussed earlier, sequencing confirmed significant differences between the 2nd ECL of VirS of CN3685 vs. CN1795. Thus, it is reasonable that, if VirS is an SP receptor then swapping expression of these virS variants between virR/S and agrB/D double null mutants of CN3685 vs, CN1795 should change their sensitivity to 8R signaling. This hypothesis was supported when the CN1795 double mutant complemented to express the CN3685 VirS variant was shown to increase its CPB production in the presence of 8R, while the CN3685 double mutant complemented to express the CN1795 VirS variant lost the ability to increase CPB production in the presence of 8R. There was some CPB production by these complementing strains even in the absence of 8R; as noted previously, this effect is likely due to overexpression of VirR from the multicopy plasmid used for complementation.

A second approach to assess whether VirS is an SP receptor used a pull-down approach to evaluate directly if VirS can physically bind SP. Because attempts to prepare a VirS antibody were unsuccessful, this pull-down experiment instead used streptavidin beads pretreated with biotin labeled 5R, which retains signaling activity. When those beads were reacted with extracts from CN3685 expressing a His₆-tagged (SEQ ID NO: 43) VirS, Western blotting with a His₆ (SEQ ID NO: 43) antibody detected specific pull-down of His₆-tagged (SEQ ID NO: 43) VirS using the biotin-labeled 5R beads. This result supported the ability of 5R to physically bind with VirS. A third and final line of evidence supporting VirS as an SP receptor came from experiments using synthetic peptides corresponding to either the ECL2 of VirS or to this ECL sequence with a single N to D substitution. The rationale behind this experiment was that if the 2nd ECL of VirS is important for SP binding, as suggested by the VirS sequencing results described earlier, then preincubating this ECL2 peptide with 5R may cause ECL2:R binding and thus inhibit subsequent Agr-like QS signaling by agrB mutants. This hypothesis was verified when preincubation of 5R with KIGK, the peptide corresponding to the wild-type ECL2 sequence of the CN1795 VirS, inhibited the ability of 5R to signal the agrB mutants of either CN3685 or CN1795, as evidenced by a reduction in 5R-induced CPB production. Furthermore, the presence of the KIGK peptide in wild-type CN3685 or CN1795 cultures also reduced CPB production. These effects were specific since KIGK_D, which is the peptide corresponding to a single N to D substitution in KIGK, did not reduce the 5R-induced increase in CPB production by the same wild-type strains or their agrB mutants. In addition to supporting SP binding to VirS, these results (as mentioned earlier) also implicate the 2nd ECL of VirS in this binding. Collectively, these ECL2 peptide results not only further support VirS:SP binding being a mechanistic basis for interactions between the Agr-like QS system and VirR/S TCRS during C. perfringens pathogenesis but could also be instructive for developing peptide therapeutics to inhibit QS signaling and reduce toxin production to control type C or type B diseases where CPB is, respectively, proven or likely to be important for virulence.

The current findings coupling the Agr-like QS system and VirS/R TCRS also hold broader relevance for understanding C. perfringens pathogenesis beyond diseases caused by type B or C strains. Both the Agr-like QS system and VirS/R TCRS positively regulate production of CPA and PFO, which are the toxins causing gas gangrene, and the importance of the Agr-like QS system for gas gangrene has been directly demonstrated. Similarly, both the Agr like QS system and VirS/R control NetB toxin production, which is required for type G strains to cause avian necrotic enteritis, and the virulence of NetB-producing type G strains requires the Agr like QS. Therefore, coupling the Agr-like QS system and VirS/R TCRS provides C. perfringens with a single, versatile regulatory pathway for controlling production of several important toxins produced during vegetative growth. However, the Agr-like QS and VirS/R systems do not universally regulate all C. perfringens toxin production during vegetative growth. For example, agrB null mutants of type B strains CN1793 and CN1795 still produce wild-type levels of ETX. Regulatory control of ETX production is poorly understood and requires further study. Coupling of the Agr-like QS and VirS/R TCRS may also be important for regulating production of toxins produced during C. perfringens sporulation since the Agr-like QS system is an important positive regulator of sporulation and C. perfringens enterotoxin (CPE) production by type F strains. However, it has not yet been assessed whether this regulation involves VirS/R.

While this study provides compelling support for VirS as an SP receptor, it remains possible that C. perfringens possesses one or more additional receptors for the SP of the Agr-like QS, particularly since this bacterium is known to possess ˜20 different TCRS. Supporting that possibility, silver staining of gels in the pull-down experiments revealed an ˜75 kDa band that was not reactive with His₆ antibody (SEQ ID NO: 43) and had a larger molecular mass (˜75 kDa) than VirS (˜50 kDa) (data not shown). Whether that band reflects nonspecific binding or a second SP receptor will require further study. In the S. aureus Agr system, AIP signaling upregulates expression of the agr operon encoding itself and the AgrC receptor in a positive feedback loop. Therefore, having obtained strong evidence supporting VirS as an SP receptor, qRT-PCR was used to examine whether the VirR/S TCRS and the Agr-like QS system upregulate expression of genes encoding each other. Results indicated that, in both CN3685 and CN1795 backgrounds, the presence of a functional virS gene results in significantly higher expression of the agrD gene and vice versa. This positive feedback effect was not attributable to growth differences between the mutants vs. their wild-type parents. These observations suggest a model where small amounts of AgrD present early during growth may help to upregulate VirS production, which then results in the availability of more receptors to further amplify AgrD production and Agr-like QS signaling later during the growth cycle. This effect could contribute to toxin production and C. perfringens pathogenicity.

### Materials and Methods

a. Bacteria, Media and Reagents.

C. perfringens wild type, null-mutant and complementing strains used in this study are listed in TABLE 3. All isolates were stored in cooked meat medium (CMM) at −20° C. Fluid thioglycolate medium (FTG, Difco Laboratories), TY broth (3% tryptic soy broth [Becton-Dickinson], 1% yeast extract [Becton Dickinson], and 0.1% sodium thioglycolate [Sigma-Aldrich]) and TGY broth (TY broth supplemented with 2% glucose [Sigma-Aldrich]) were used for broth cultures. After inoculation, Brain Heart Infusion (BHI) agar (Research Products International) plates containing 15 μg ml⁻¹ chloramphenicol (Sigma-Aldrich) were incubated at 37° C. under anaerobic growth conditions in Gas-Pak jars to screen the knockout mutants constructed in this study. A CN3685::agrB null mutant named BMJV10, an CN1795::agrB null mutant, and complementing strains of both mutants had been previously constructed and characterized. All chemical reagents used in this study were purchased from Fisher Scientific, Sigma Aldrich or Bio-Rad Laboratories.

TABLE 3
C. perfringens strains used in Example II

| | | |
|---|---|---|
| | | Description (disease, location/date |
| No. | Isolates | of isolation) |

Type B

| | | |
|---|---|---|
| 1 | CN1795 | Veterinary lab, toxigenic, 1947 |
| 2 | CN1795::agrB | CN1795 agrB null mutant |
| 3 | CN1795::agrBcomp | CN1795 agrB null mutant complemented |
| | | with agr operon |
| 4 | CN1795::virS | CN1795 virS null mutant |
| 5 | CN1795::virS cN1795 | CN1795 virS null mutant complemented |
| | virR/S | with CN1795 virR/S operon |
| 6 | CN1795::virSc3685 | CN1795 virS null mutant complemented |
| | virR/S | with CN3685 virR/S operon |
| 7 | CN1795DKO | CN1795 virS/agrB double null mutant |
| 8 | CN1795DKOc3685 | CN1795 virS/agrB double null mutant |
| | virR/S | complemented with CN3685 virR/S |
| | | operon |

Type C

| | | |
|---|---|---|
| 1 | CN3685 | Sheep with struck |
| 2 | CN3685::agrB | CN3685 agrB null mutant |
| | (BMJV10) | |
| 3 | CN3685::agrBcomp | CN3685 agrB null mutant complemented |
| | | with agr operon |
| 4 | CN3685::virS | CN3685 virS null mutant |
| 5 | CN3685::virSc1795 | CN3685 virS null mutant complemented |
| | virR/S | with CN1795 virR/S operon |
| 6 | CN3685::virSc3685 | CN3685 virS null mutant complemented |
| | virR/S | with CN3685 virR/S operon |
| 7 | CN3685DKO | CN3685 virS/agrB double null mutant |
| 8 | CN3685DKOc1795 | CN3685 virS/agrB double null mutant |
| | virR/S | complemented with CN1795 virR/S |
| | | operon |

b. Synthetic Peptides.

The synthesis of all peptides used in this study was carried out by the Peptide and Peptoid Synthesis Core Facility Division of the Health Sciences Core Research Facilities (HSCRF) at the University of Pittsburgh. SP-based synthetic peptides used included: i) 5R, a 5-mer cyclic ring likely corresponding to the wild-type C. perfringens SP; ii) 8R, an 8-mer consisting of 5R plus a three amino acid tail, and iii) Bio-5R, a biotin-labeled version of 5R. Without being bound by theory, there is evidence to suggest that the structures of certain AIPs previously believed to exist as thiolactones, such as the 5-mer thiolactone ring initially produced by C. perfringens, may be cysteine-containing homodetic peptides in their functional state. These peptides were prepared according to known methods. Synthesis of the linear VirS-based peptides KIGK and KIGK_D (TABLE 2) were performed on a 50 μM scale using standard FMOC (9-fluorenylemethyloxycarbonyl) chemistry cycles on a Liberty Blue CEM microwave synthesizer using Oxyma/DIC (Ethyl-(2Z)-2-cyano-2-hydroxyiminiacetate/N,N Diisopropylcarbodiimide) activation in Dimethylforamide (DMF). FMOC-protected amino acids (EMD 475 Millipore) were used for the stepwise assembly of these linear sequences on pre-loaded FMOC-Lys(BOC) Wang resin (0.56 mmole, Peptides International). A cleavage cocktail (90% Trifluoroacetic acid (TFA), 5% Thioanisole, 3% 3,6-Dioxa-1,8-octanedithiol (DODT), 2% Anisole) was used for 4 h at room temperature, while shaking, to cleave KIGK and KIGK_D from the Wang resin as well as to scavenge side-chain protecting groups. These peptides were first precipitated in cold diethyl ether and then washed and centrifuged 3 times with additional diethyl ether resulting in a pellet of crude peptide. Crude KIGK and KIGK_D were allowed to air dry and were then dissolved in 0.1% TFA which was then frozen and lyophilized overnight to remove remaining organics. Each crude peptide was dissolved in 0.1% TFA and then directly loaded onto a Waters 2555 Quaternary Gradient Module with a Waters 2489 UV-Vis Detector and purified on a Phenomenex Gemini (250×21.20 mm) 10 μm C-18 column using standard Acetonitrile (ACN)/0/1% TFA gradient conditions. Final analytical determinations of peptide purity for KIGK and KIGK_D were performed on a Waters e2695 Separations Module with a Waters 2489 UV-Vis detector using a Phenomenex Gemini-NX (250×4.6 mm) 5 μm C-18 column using standard Acetonitrile (ACN)/0.1% TFA gradient conditions. Mass spectrometry analysis using an Applied Biosystems Voyager DE491 STR MALDI-TOF was used to confirm the expected mass of each peptide: KIGK expected, 1513.80; observed, 1513.04; KIGK_D expected, 1514.78; observed 1514.06.

Each synthetic peptide was resuspended in DMSO (Fisher Scientific) at 50 mM before use. Aliquots (2.0 μl, 10 μl or 20 μl) of this suspension of synthetic peptide in DMSO were then added to 1 ml of culture medium, resulting in a final peptide concentration of 100 μM, 500 μM or 1 mM. After a 5 h or overnight (16 h) culture, CPB or His-tag Western blots were performed using culture supernatants or pelleted cells (See Western blot analyses section).

c. Sequencing of the virR/S Operon in CN1795 and CN3685.

DNA was isolated from CN1795 or CN3685 using the Master Pure Gram-positive DNA purification kit (Epicentre). The primers used in a PCR to amplify this operon are listed in TABLE 4. For this PCR reaction, 0.25 μl of each primer (at a 25 μM final concentration), 1 μl of purified DNA template and 25 μl NEBNext® High-Fidelity 2×PCR Master mix (New England Biolabs) were mixed together and double-distilled water (ddH₂O) was then added to reach a total reaction volume of 50 μl. The reaction mixtures were placed in a thermal cycler (Techne) and then subjected to the following amplification conditions: 1 cycle of 98° C. for 30 sec; 35 cycles of 98° C. for 10 s, 50-72° C. (depending on the primers used) for 20 s, and 72° C. for 2 min; and a single extension of 72° C. for 2 min. The resultant PCR products were cleaned-up using a QIAquick PCR purification kit (Qiagen) and sent for DNA sequencing to the University of Pittsburgh Core Sequencing Facility. The sequence results were submitted to NIH gene bank (accession numbers are MT597430 and MT597431).

TABLE 4
Primers used in Example II.

| | | | | PCR |
|---|---|---|---|---|
| | | SEQ ID | | product |
| Primer′s name | Primer′s sequence | NO: | Purpose | size (bp) |
| virS-293\|294a- | AAAAAAGCTTATAATTATCCT | 15 | pJIR750virSi | 350 |
| IBS | TATCTACCGTAAGCGTGCGCC | | construction | |
| | CAGATAGGGTG | | | |
| virS-293\|294a- | CAGATTGTACAAATGTGGTGA | 16 | | |
| EBS1d | TAACAGATAAGTCGTAAGCAT | | | |
| | TAACTTACCTTTCTTTGT | | | |
| virS-293\|294a- | TGAACGCAAGTTTCTAATTTC | 17 | | |
| EBS2 | GGTTGTAGATCGATAGAGGAA | | | |
| | AGTGTCT | | | |
| EBS universal | CGAAATTAGAAACTTGC | 18 | | |
| | GTTCAGTAAAC | | | |
| virSKOF | TAAGTCAATTTAGCCCTAAGA | 19 | Screen for | 376 |
| | AAA | | intron | |
| virSKOR | CGAAACTTTAAACATCTAACA | 20 | insertion in | |
| | ACCA | | virS | |
| | | | virS RT-PCR | |
| NagrBKOF | TGGAACTTATGCTCTAATACA | 21 | Screen for | 536 |
| | AACA | | intron | |
| agrBKOR | AATCTATAGTTTTTAACAATAT | 22 | insertion in | |
| | ATTT | | agrB | |
| | | | agrB RT-PCR | |
| cpbF | GCGAATATGCTGAATCATCTA | 23 | cpb qRT-PCR | 196 |
| cpbR | GCAGGAACATTAGTATATCTT | 24 | | |
| | C | | | |
| qvirSF | CATAGCCTGTATTGAAGGAAA | 25 | virS qRT-PCR | 229 |
| | TAAC | | | |
| qvirSR | TGTGCAGATATCAAAGTACTC | 26 | | |
| | A | | | |
| qvirRF | CCTTTGAGACAGGAGAGGATC | 27 | virR qRT-PCR | 240 |
| | TA | | | |
| qvirRR | CCTGCTCTTGTAGCTCCTTAAA | 28 | | |
| | T | | | |
| qagrDF | GCTGCATTAACAACAGTAGTT | 29 | agrD qRT- | 76 |
| | GC | | PCR | |
| qagrDR | GTTCCTCTGGTTGGTGTGTAA | 30 | | |
| | A | | | |
| VirR/SseqF | GTCTCAAAGATCTAGTAAAAT | 31 | virR/S operon | 838 |
| | GGGA | | sequencing | |
| VirR/SseqR | GCCCTTATTATTAAGCTCCTTT | 32 | | |
| | TCT | | | |
| VirR/Sseq1 | CAGGTTACAGCTTGTGTAGAA | 33 | | 912 |
| | AATA | | | |
| VirR/Sseq1R | CCTTAAAGGCATATCCAAATA | 34 | | |
| | TAAC | | | |
| VirR/Sseq2 | CAATATAAAATGTATTATGAT | 35 | | 1050 |
| | CTC | | | |
| VirR/Sseq2R | GGAATGAGCATTTTTAATATG | 36 | | |
| | AATT | | | |
| VirR/Sseq3 | GACAAGCTAAACTTAGGATT | 37 | | 813 |
| VirR/Sseq3R | TTCCATTTACCTGAATTAACTC | 38 | | |
| | ACT | | | |
| VirR/ScompF | CCGGGGATCCGAAAGTGGATA | 39 | virR/S | 2494 |
| | TGCACTAGGAAC | | complementation | |
| VirR/ScompR | ATGCCTGCAGTGCAAAGCTTA | 40 | | |
| | AAACTGTAACTGTA | | | |
| VirR/ShisR | ATGCCTGCAGTTAATGATGAT | 41 | virR/S-His tag | 2249 |
| | GATGATGATGGGCTTCTTTTTC | | complementation | |
| | TTGATTTATAGG | | | |

d. Computer Modeling of the VirS Protein Structure.

The virS sequencing results were used to model the predicted structure of VirS made by CN1795 or CN3685 using the TMHMM server v.2.0 (prediction of transmembrane helices in proteins (www.cbs.dtu.dk/services/TMHMM/) transmembrane prediction algorithms.

e. Plasmids and Primers.

A plasmid named pJIR750virSi was constructed to inactivate, using Clostridium-modified group II TargeTron® Technology, the virS gene and create single virS null mutants or, using existing agrB mutants, double mutants unable to express both virS and the operon encoding AgrB/D. The primers used for intron targeting to the virS gene were virS25 293|294a-IB S, virS-293|294a-EBS1d, virS-293|294a-EBS2 and EBS universal primers. All primer sequences are listed in TABLE 4. The 350-bp intron PCR product was then inserted into pJIR750ai between the HindIII and BsrGI enzyme (New England Biolabs) cut sites to construct the pJIR750virSi vector. The virS null mutant screening primers used were virSKOF and virSKOR. The same pair of primers were also employed to analyze virS gene expression. The primers used for PCR amplification of the agrB gene or for agrB gene RT-PCR were NagrBKOF and agrBKOR. The qRT-PCR primers used in this study were designed and synthesized by Integrated DNA Technologies (IDT). These primers were specific for 16S RNA (as a control housekeeping gene), the virS gene (qvirSF and qvirSR); or the agrD gene (qagrDF and qagrDR).

A virR/S operon complementation vector, named pJIR750virR/Scomp, was constructed as follows. A region of CN1795 or CN3685 DNA spanning from ˜200 bp upstream of the virR/S operon to ˜200 bp downstream of the virR/S operon was PCR amplified using primers VirR/ScompF and VirR/ScompR (TABLE 4). The resultant PCR product (3000 bp) was then ligated into pJIR750 between the BamHI and PstI restriction sites, followed by electroporation of the virR/S operon-carrying plasmid pJIR750virR/Scomp into the CN1795::virS, CN3685::virS, CN1795DKO or CN3685DKO mutants (see Results). Transformants were then selected on BHI agar plates containing 15 μg mL⁻¹ of chloramphenicol with anaerobic incubation in Gas-Pak jars.

For expression of His₆-tagged (SEQ ID NO: 43) VirS, the virR/S operon was PCR amplified using primers VirR/ScompF and VirR/ShisR (TABLE 4), which added a His₆ sequence (SEQ ID NO: 43) to the C-terminal end of the VirS membrane protein. The resultant PCR product was then ligated into pJIR750 between the BamHI and PstI restriction sites to create the plasmid pJIR750virS/hiscomp. This plasmid was then electroporated into the CN3685::virS to create a complementing strain (CN3685::virSc3685virShis) that expresses VirS protein labeled with a His₆-tag (SEQ ID NO: 43).

g. Construction of Single CN1795 and CN3685 virS Null Mutants and Complemented Strains, or Double Null Mutants of Those Strains with Inactivated virS and agrB Genes.

The virS gene was disrupted in CN1795 or CN3685 to generate single null mutant strains that do not produce VirS. The virS gene was also inactivated in existing CN1795::agrB or CN3685::agrB strains to create double null mutants with inactivated virS and agrB genes. Disruption of the virS gene in these mutants was achieved by specifically inserting, in the antisense orientation, a group II intron (˜900 bp) into the virS gene, generating single virS null mutants, or double virS/agrB mutants, of CN1795 or CN3685. For this purpose, the intron donor plasmid pJIR750virSi, which carries a virS-targeted intron, was electroporated into CN1795, CN3685, CN1795::agrB or CN3685::agrB. Transformants were selected by plating onto BHI agar plates containing 15 μg ml⁻¹ of chloramphenicol, followed by overnight anaerobic growth in a Gas-Pak jar. Colony PCR was carried out for screening using internal virS primers virSKOF and virSKOR (TABLE 2), which amplify a PCR product of ^{˜}370 bp using DNA from a wild-type strain but amplify an ˜1300 bp product using DNA from virS null mutants due to the insertion of a ^{˜}900 bp intron. Each virS gene null mutant was subcultured daily in FTG medium over 10 days to cure the intron carrying plasmid, creating a CN1795 virS null mutant (CN1795::virS), a CN3685 virS null mutant (CN3685::virS), a CN1795 virS/agrB double null mutant (CN1795DKO) and a CN3685 virS/agrB double null mutant (CN3685DKO). Each mutant was then further characterized by PCR, RT-PCR and Southern blotting analyses, as described below.

VirS complementing strains of the single and double mutants were prepared by transformation with pJIR750virR/S1795 comp or pJIR750virR/53685comp. Transformants were selected on chloramphenicol as described earlier.

h. Measurement of C. perfringens Growth.

For analysis of C. perfringens vegetative growth, a 0.2-mL aliquot of an overnight FTG culture of a wild-type or null mutant strain was inoculated into 10 mL of TY medium. The cultures were incubated at 37° C.; thereafter, at 0, 1, 3, 5, 8 and 24 h culture times, 1-mL of each culture was removed for optical density measurement at 600 nm (OD₆₀₀) using a Biorad Smart spectrometer.

For the two wild-type strains, another 1-mL aliquot culture was removed and centrifuged at 15,000 rpm for 3 min Equal volumes of each resultant culture supernatant was then mixed with 5×SDS loading buffer and boiled for 5 min Thirty microliters of each boiled sample were electrophoresed on a 10% SDS-PAGE gel and then subjected to a CPB Western blot (See Western blot analyses below). Using the same cultures, total RNA was isolated from the pellets at each timepoint and qRT-PCR were performed as described in the RNA, RT-PCR and qRT-PCR section.

i. C. perfringens DNA Isolation, PCR and Intron Southern Blot Analyses.

DNA was extracted from all C. perfringens strains using the MasterPure Gram-Positive DNA purification kit. PCR for the virS or agrB genes was performed using the primers described in the previous section. For the wild-type strains, the sizes of PCR products are listed in TABLE 4. For the null mutant strains, PCR using the same pair of the primers should amplify a product from the intron-disrupted gene that is ˜900 bp larger compared to the gene from the wild-type strains. Suitable PCR conditions are known in the art.

For Southern blot analysis to detect an intron insertion, aliquots (3 μg each) of wild-type, single or double null mutant strain DNA were digested overnight with EcoRI at 37° C. according to the manufacturer's instructions (New England Biolabs). The digested DNA samples were then electrophoresed on a 1% agarose gel before transfer onto a positively-charged nylon membrane (Roche) for hybridization with an intron-specific probe. The intron specific probe was prepared using the PCR DIG Probe Synthesis Kit (Roche) and intron primers (IBS and EBS2). After hybridization, Southern blots were developed using reagents from the DIG DNA labeling and detection kit (Roche), according to the manufacturer's instructions.

j. C. perfringens RNA Isolation, RT-PCR and Quantitative Reverse Transcription PCR (QRT PCR) Analyses.

All tested isolates were grown in TY broth for 2 h (for analyzing virS expression) or 5 h (for analyzing expression of agrD and agrB) at 37° C. Cultures were then pelleted at the indicated times and RNA was extracted using saturated phenol and purified by TRIzol and chloroform (Life Technology and Sigma), according to known methods. Before RT-PCR or qRT PCR were performed, the isolated RNA was subjected to regular PCR without reverse transcriptase to confirm that samples were free of DNA. If the sample had any DNA contamination, DNase (Thermo Fisher) was used to remove the residual DNA. RNA was then quantified by determining the absorbance at 260 nm. The purified RNA was used to prepare cDNA or stored in a −80° C. freezer for further experiments.

A 1 μl aliquot of purified RNA (100 ng) was used in a one-step RT-PCR containing 10 μl of 2×Taq Master Mix (New England Biolabs), avian myeloblastosis virus (AMV) reverse transcriptase (4 U; Promega), ddH2O, and primers specific for the virS or agrB genes (TABLE 4), 16S RNA RT-PCR was performed to serve as a loading control. Reaction mixtures were incubated for 45 min at 45° C. to allow cDNA synthesis, then regular PCR cycling was performed as follows: (i) 95° C. for 2 min; (ii) 30 cycles of 95° C. for 15s, 50° C. for 30s, and 68° C. for 30s; and (iii) a final extension of 68° C. for 5 min.

For qRT-PCR, an aliquot (500 ng) of purified RNA in a total 10 μl reaction mixture was first synthesized to cDNA using a Maxima first-strand cDNA synthesis kit (Thermo Scientific), according to the manufacturer's instructions. The cDNA synthesis programming was: 25° C. for 10 min, 50° C. for 30 min and 85° C. for 5 min. All qRT-PCR primers were designed using the Integrated DNA Technologies (IDT) website and listed in TABLE 4. Each cDNA was diluted 10 times to 5 ng/μl. Power SYBR green PCR master mix (Thermo Fisher Scientific) and a StepOnePlus qRT-PCR instrument (Applied Biosystems) were used to perform qRT-PCR, according to previously disclosed methods. After qRT-PCR, the relative quantitation of mRNA expression was normalized to the level of constitutive expression of the housekeeping 16S RNA and calculated by the comparative threshold cycle (C_{T}; 2^{-ΔΔCT}) method.

k. Use of SP-Based Peptides to Induce CPB Production.

Wild-type or agrB mutants of CN1795 and CN3685 were each grown in 10 ml of FTG overnight at 37° C. An aliquot (0.2 ml) of each culture was inoculated into 10 ml of fresh TY broth, and those cultures were incubated overnight (about 16 h) at 37° C. A 15-μl aliquot of each TY overnight culture was then inoculated into 1 ml of TY medium in 1.5 ml microcentrifuge tube with a screw cap (Fisher Scientific) that contained a final concentration of 100 μM of an SP-based peptide (5R, 8R of B-5R) suspended in DMSO. As a control, an equal volume of DMSO alone (no peptide) was added to some cultures. These cultures were incubated for 5 h or overnight at 37° C. as specified in the experiments.

### 1. Use of VirS ECL2-Based Peptides to Inhibit CPB Signaling.

CN1795 and CN3685 agrB null mutant strains were each grown in 10 ml of FTG overnight at 37° C. An aliquot (0.2 ml) of each culture was inoculated into 10 ml of fresh TY broth and those cultures 635 were incubated overnight (about 16 h) at 37° C. Before the addition of a 15-μl aliquot of each TY overnight culture, 5R (100 μM final concentration) together with a 500 μM final concentration of the 14 Aa-peptide (KIGK) corresponding to the VirS ECL2 sequence, the variant peptide (KIGK_D), or a control peptide was dissolved in DMSO and the mixed in 1 ml TY medium and incubated at 37° C. for 30 mM. A 15-μl aliquot of the overnight agrB null mutant culture was inoculated into this medium and incubated for 5 h. At that time, the culture supernatants were collected and used for CPB Western blot analysis.

In a second experiment, wild-type CN1795 and CN3685 were each grown in 10 ml of FTG overnight at 37° C. An aliquot (0.2 ml) of each culture was inoculated into 10 ml of fresh TY broth, and those cultures were incubated overnight (about 16 h) at 37° C. A 15-μl aliquot of each TY overnight culture was then inoculated into 1 ml of TY medium in a 1.5 ml microcentrifuge tube with a screw cap that contained a final concentration of 500 μM or 1 mM of KIGK or KIGK_D dissolved in DMSO. These cultures were then incubated for 5 h. At that time, the culture supernatants were collected and used for CPB Western blot analysis.

m. Western Blot Analyses of CPB Production.

Aliquots of each culture were adjusted to equal OD₆₀₀ values and then centrifuged. The supernatants were mixed with 5×SDS-PAGE loading buffer and boiled for 5 min. Twenty microliters of each sample were electrophoresed on a 10% SDS-PAGE gel and the separated proteins were then transferred onto a nitrocellulose membrane. The membrane was blocked with TBS-Tween 20 (0.05% v/v) and non-fat dry milk (5% w/v) for 1 h at room temperature, followed by probing with a rabbit poly-anti-CPB antibody (1:1000 dilution) overnight at 4° C. Finally, bound antibody was detected with a horseradish 657 peroxidase-conjugated secondary anti-rabbit antibody (Sigma Aldrich), followed by addition of SuperSignal West Pico Chemiluminescent Substrate (Fisher Scientific).

n. His₆-Tagged (SEQ ID NO: 43) VirS Pull-Down by Bio-5R and Detection by Western Blot.

CN3685::virS was transformed with a complementation vector named pJIR750virR/Shiscomp, which encodes a His₆-tagged (SEQ ID NO: 43) VirS (See Plasmids and primers section), to create CN3685::virSc3685virR/Shis. As an antibody specificity control, CN3685::virS was also transformed to create a strain named CN3685::virSc3685virR/S that expresses untagged VirS. A 0.2 ml aliquot of a CMM stock of each culture was transferred to 10 ml FTG medium and then cultured overnight at 37° C. A 0.2 ml aliquot of each FTG culture was transferred to 10 ml TY medium for another overnight culture at 37° C. A 2 ml aliquot of this overnight TY culture was transferred to 100 ml of fresh TY medium and cultured for 4 h at 37° C. Those cultures were then centrifuged and the pellets frozen at −80° C. for at least 2 h. The frozen pellets were resuspended in 1 ml of B-per buffer (Fisher Scientific) with proteinase inhibitor (Research Products International) at room temperature for 30 min with gentle shaking. At the same time, a 100 μl aliquot of Streptavidin Magbeads (Genscript) was washed twice with B-per buffer. The washed beads were then incubated with either biotinylated 5R (Bio-5R) or control 5R in 500 μl B-per buffer (200 μM) for 1 h at room temperature with slow end over-end mixing. These pretreated beads were then washed twice with B-per buffer. The bacterial supernatants, prepared as described above, were applied to the washed Magbeads and the mixture was incubated at 4° C. overnight with slow end-over-end mixing. The Magbeads was washed three times and then 80 μl of 2×SDS loading dye was added, followed by boiling for 5 min. A 40 μl aliquot of each sample was loaded on 10% SDS gel, followed by a His₆-tag (SEQ ID NO: 43) detection Western blot.

For this purpose, a His₆-Tag antibody (SEQ ID NO: 43) (Aviva Systems Biology OAEA00010) was purchased from Fisher Scientific. The antibody was used at a 1:1000 dilution in TTBS with 5% milk. Finally, bound antibody was detected with a horseradish peroxidase-conjugated secondary anti-mouse antibody (Sigma Aldrich), followed by addition of Clarity and Clarity Max ECL Western Blotting Substrates (Bio-rad).

o. Mice Inoculation

20-25 gr BalbC mice of either gender were inoculated intramuscularly in the thigh with each of the strains used for this project. They were examined regularly during 4 hours after which they were euthanized. Gross lesions were recorded and samples were collected from the inoculated thigh and the contralateral thigh (control). These samples were processed for histology and microscopic examination. Additional samples were subjected to anaerobic culture and colony count.

p. Statistical Analyses

All statistical analyses were performed using GraphPad Prism 8. For comparison of more than two samples, one-way analysis of variance (ANOVA) was applied with post hoc analysis by Dunnett's multiple-comparison test. For comparison of two samples, student T-test was applied. Differences were considered significant when the P value was less than 0.05.

q. Determining Effects of the KIGK Synthetic Peptide on C. perfringens-Induced Lethality in vivo.

Since the KIGK peptide was able to reduce in vitro production of CPB, the VirS 2nd ECL-based peptide will be further evaluated for its ability to reduce toxin production in vivo. For example, determining whether KIGK affects virulence induced by C. perfringens infection can be studied using a rabbit small intestinal loop model or using intragastrically and intraduodenally challenged mice, which are known in the art for investigating lethal enterotoxemias induced by CPB toxin. The effect of KIGK administration on NetB toxin production can be studied in vivo using an experimentally-induced avian necrotic enteritis model. In addition, reduction of CPA and PFO production by KIGK can be studied in the gas gangrene mouse model described above for the 6-R peptide.

Other embodiments of the present disclosure are possible. Although the description above contains specific examples, these should not be construed as limiting the scope of the disclosure. Features and aspects of the disclosed embodiments can be combined with or substituted for one another in order to form various embodiments. Thus, the scope of the present disclosure should not be limited by a particular embodiment described above. Rather, the scope of this disclosure should be determined by the appended claims and their legal equivalents.

Reference to an element in the singular is not intended to mean “one and only one” unless explicitly so stated. All structural, chemical, and functional equivalents to the elements of the above-described preferred embodiment that are known to those of ordinary skill encompassed by the present claims. No element, component, or method step in the present disclosure is intended to be dedicated to the public regardless of whether the element, component, or method step is explicitly recited in the claims.

What is claimed is:

1. A medicament comprising an Agr-like QS system inhibitor of Clostridium perfringens and a pharmaceutically acceptable carrier, wherein the inhibitor is a 6-R peptide, a C. perfringens Signaling Peptide (SP) receptor peptidomimetic, or a combination thereof.

2. The medicament of claim 1, wherein the inhibitor comprises a VirS-based SP receptor peptidomimetic, optionally a peptide having an amino acid sequence consisting of the sequence set forth in SEQ ID NO: 13 or SEQ ID NO: 10.

3. The medicament of claim 1, wherein the inhibitor is in a salt, solvate, or prodrug form.

4. The medicament of claim 1 formulated to be administered to a subject orally, buccally, sublingually, nasally, intravenously, intramuscularly, intrathecally, intraperitoneally, transdermally, or by pulmonary administration.

5. The medicament of claim 1, comprising 25 to 100 μM the Agr-like QS system inhibitor in a liquid carrier.

6. A food or animal feed comprising an Agr-like QS system inhibitor of Clostridium perfringens and an edible carrier, wherein the inhibitor is a 6-R peptide, a C. perfringens Signaling Peptide (SP) receptor peptidomimetic, or a combination thereof.

7. The food or animal feed of claim 6, wherein the inhibitor comprises a VirS-based SP receptor peptidomimetic, optionally a peptide having an amino acid sequence consisting of the sequence set forth in SEQ ID NO: 13 or SEQ ID NO: 10.

8. The food or animal feed of claim 6, wherein the food or feed comprises beef, poultry, gravies, or dried or pre-cooked food.

9. A method of preventing or treating a disease associated with a Clostridium perfringens infection or infection by other pathogenic clostridia having a Agr-like QS system comprising administering, to a subject in need of prevention or treatment, a therapeutically effective amount of an Agr-like QS system inhibitor of C. perfringens selected from a 6-R peptide, a C. perfringens Signaling Peptide (SP) receptor peptidomimetic, or a combination thereof.

10. The method of claim 9, wherein the inhibitor comprises a VirS-based SP receptor peptidomimetic.

11. The method of claim 10, wherein the VirS-based SP receptor peptidomimetic is a peptide having an amino acid sequence consisting of the sequence set forth in SEQ ID NO: 13 or SEQ ID NO: 10.

12. The method of claim 9, wherein the subject is selected from the group consisting of primates, rodents, domestic animals and game animals, optionally wherein the domestic animals include cows, horses, pigs, or chicken.

13. The method of claim 9, wherein the inhibitor is administered with a food selected from the group consisting of beef, poultry, gravies, and dried or pre-cooked foods.

14. The method of claim 9, wherein the inhibitor is administered with an antibiotic effective for treating a Clostridium infection.

15. The method of claim 9, wherein the method further comprises debridement or removal of necrotic tissue caused by the infection.

16. The method of claim 9, wherein the therapeutically effective amount reduces production of at least one of alpha toxin (CPA), beta toxin (CPB), epsilon toxin (ETX), iota toxin (ITX), perfringolysin O (PFO), enterotoxin (CPE), NetB toxin (NetB) or beta2 toxin (CPB2) in an infected tissue of the subject as compared to production of the toxin in a corresponding tissue of an untreated subject infected with the C. perfringens bacteria.

17. The method of claim 9, wherein the therapeutically effective amount prevents or reduces swelling or hemorrhage in an infected tissue of the subject as compared to a corresponding tissue of an untreated subject infected with the C. perfringens bacteria.

18. The method of claim 9, wherein the therapeutically effective amount prevents or reduces at least one of muscle degeneration, necrosis and inflammation in an infected tissue of the subject as compared to a corresponding tissue of an untreated subject infected with the C. perfringens bacteria.

19. The method of claim 9, wherein the disease is necrotic enteritis, gas gangrene, enterotoxemia or enteritis.

20. The method of claim 9, wherein the inhibitor is administered by intramuscular injection at a concentration of 25 to 100 μM.


SEQUENCE LISTING (43 sequences)

SEQ ID NO: 1 - protein, 5 aa, Clostridium perfringens
CLWFT

SEQ ID NO: 2 - protein, 6 aa, Artificial Sequence
  Description of Artificial Sequence Synthetic peptide
CLWFTH

SEQ ID NO: 3 - DNA, 53 bp, Artificial Sequence
  Description of Artificial Sequence Synthetic primer
aaaaaagcttataattatccttagtgttcattggagtgcgcccagatagggtg

SEQ ID NO: 4 - DNA, 60 bp, Artificial Sequence
  Description of Artificial Sequence Synthetic primer
cagattgtacaaatgtggtgataacagataagtcattggaattaacttacctttctttgt

SEQ ID NO: 5 - DNA, 49 bp, Artificial Sequence
  Description of Artificial Sequence Synthetic primer
tgaacgcaagtttctaatttcgattaacactcgatagaggaaagtgtct

SEQ ID NO: 6 - DNA, 25 bp, Artificial Sequence
  Description of Artificial Sequence Synthetic primer
tggaacttatgctctaatacaaaca

SEQ ID NO: 7 - DNA, 26 bp, Artificial Sequence
  Description of Artificial Sequence Synthetic primer
aatctatagtttttaacaatatattt

SEQ ID NO: 8 - DNA, 20 bp, Artificial Sequence
  Description of Artificial Sequence Synthetic primer
gctaatgttactgccgttga

SEQ ID NO: 9 - DNA, 20 bp, Artificial Sequence
  Description of Artificial Sequence Synthetic primer
cctctgatacatcgtgtaag

SEQ ID NO: 10 - protein, 14 aa, Artificial Sequence
  Description of Artificial Sequence Synthetic peptide
KINSLVNVSELLGK

SEQ ID NO: 11 - protein, 14 aa, Artificial Sequence
  Description of Artificial Sequence Synthetic peptide
AYSSGAPPMPPFFF

SEQ ID NO: 12 - protein, 14 aa, Artificial Sequence
  Description of Artificial Sequence Synthetic peptide
KINSLVDVSELLGK

SEQ ID NO: 13 - protein, 14 aa, Artificial Sequence
  Description of Artificial Sequence Synthetic peptide
  MOD_RES (4)..(4): S or N
  MOD_RES (6)..(6): V or S
  MOD_RES (7)..(7): N or S
  MOD_RES (8)..(8): V or I
  MOD_RES (10)..(10): E or D
KINXLXXXSXLLGK

SEQ ID NO: 14 - protein, 6 aa, Clostridium perfringens
RVDIGI

SEQ ID NO: 15 - DNA, 53 bp, Artificial Sequence
  Description of Artificial Sequence Synthetic primer
aaaaaagcttataattatccttatctaccgtaagcgtgcgcccagatagggtg

SEQ ID NO: 16 - DNA, 60 bp, Artificial Sequence
  Description of Artificial Sequence Synthetic primer
cagattgtacaaatgtggtgataacagataagtcgtaagcattaacttacctttctttgt

SEQ ID NO: 17 - DNA, 49 bp, Artificial Sequence
  Description of Artificial Sequence Synthetic primer
tgaacgcaagtttctaatttcggttgtagatcgatagaggaaagtgtct

SEQ ID NO: 18 - DNA, 28 bp, Artificial Sequence
  Description of Artificial Sequence Synthetic primer
cgaaattagaaacttgcgttcagtaaac

SEQ ID NO: 19 - DNA, 24 bp, Artificial Sequence
  Description of Artificial Sequence Synthetic primer
taagtcaatttagccctaagaaaa

SEQ ID NO: 20 - DNA, 25 bp, Artificial Sequence
  Description of Artificial Sequence Synthetic primer
cgaaactttaaacatctaacaacca

SEQ ID NO: 21 - DNA, 25 bp, Artificial Sequence
  Description of Artificial Sequence Synthetic primer
tggaacttatgctctaatacaaaca

SEQ ID NO: 22 - DNA, 26 bp, Artificial Sequence
  Description of Artificial Sequence Synthetic primer
aatctatagtttttaacaatatattt

SEQ ID NO: 23 - DNA, 21 bp, Artificial Sequence
  Description of Artificial Sequence Synthetic primer
gcgaatatgctgaatcatcta

SEQ ID NO: 24 - DNA, 22 bp, Artificial Sequence
  Description of Artificial Sequence Synthetic primer
gcaggaacattagtatatcttc

SEQ ID NO: 25 - DNA, 25 bp, Artificial Sequence
  Description of Artificial Sequence Synthetic primer
catagcctgtattgaaggaaataac

SEQ ID NO: 26 - DNA, 22 bp, Artificial Sequence
  Description of Artificial Sequence Synthetic primer
tgtgcagatatcaaagtactca

SEQ ID NO: 27 - DNA, 23 bp, Artificial Sequence
  Description of Artificial Sequence Synthetic primer
cctttgagacaggagaggatcta

SEQ ID NO: 28 - DNA, 23 bp, Artificial Sequence
  Description of Artificial Sequence Synthetic primer
cctgctcttgtagctccttaaat

SEQ ID NO: 29 - DNA, 23 bp, Artificial Sequence
  Description of Artificial Sequence Synthetic primer
gctgcattaacaacagtagttgc

SEQ ID NO: 30 - DNA, 22 bp, Artificial Sequence
  Description of Artificial Sequence Synthetic primer
gttcctctggttggtgtgtaaa

SEQ ID NO: 31 - DNA, 25 bp, Artificial Sequence
  Description of Artificial Sequence Synthetic primer
gtctcaaagatctagtaaaatggga

SEQ ID NO: 32 - DNA, 25 bp, Artificial Sequence
  Description of Artificial Sequence Synthetic primer
gcccttattattaagctccttttct

SEQ ID NO: 33 - DNA, 25 bp, Artificial Sequence
  Description of Artificial Sequence Synthetic primer
caggttacagcttgtgtagaaaata

SEQ ID NO: 34 - DNA, 25 bp, Artificial Sequence
  Description of Artificial Sequence Synthetic primer
ccttaaaggcatatccaaatataac

SEQ ID NO: 35 - DNA, 24 bp, Artificial Sequence
  Description of Artificial Sequence Synthetic primer
caatataaaatgtattatgatctc

SEQ ID NO: 36 - DNA, 25 bp, Artificial Sequence
  Description of Artificial Sequence Synthetic primer
ggaatgagcatttttaatatgaatt

SEQ ID NO: 37 - DNA, 20 bp, Artificial Sequence
  Description of Artificial Sequence Synthetic primer
gacaagctaaacttaggatt

SEQ ID NO: 38 - DNA, 25 bp, Artificial Sequence
  Description of Artificial Sequence Synthetic primer
ttccatttacctgaattaactcact

SEQ ID NO: 39 - DNA, 33 bp, Artificial Sequence
  Description of Artificial Sequence Synthetic primer
ccggggatccgaaagtggatatgcactaggaac

SEQ ID NO: 40 - DNA, 35 bp, Artificial Sequence
  Description of Artificial Sequence Synthetic primer
atgcctgcagtgcaaagcttaaaactgtaactgta

SEQ ID NO: 41 - DNA, 55 bp, Artificial Sequence
  Description of Artificial Sequence Synthetic primer
atgcctgcagttaatgatgatgatgatgatgggcttctttttcttgatttatagg

SEQ ID NO: 42 - protein, 8 aa, Artificial Sequence
  Description of Artificial Sequence Synthetic peptide
TSACLWFT

SEQ ID NO: 43 - protein, 6 aa, Artificial Sequence
  Description of Artificial Sequence Synthetic 6xHis tag
HHHHHH